(19) United States
(12) Patent Application Publication
(10) Pub. No.: US 2019/0204341 A1
(43) Pub. Date: Jul. 4, 2019

(54) COMPOSITIONS AND METHODS FOR DIAGNOSING CELIAC DISEASE USING CIRCULATING CYTOKINES/CHEMOKINES

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 16/311,805

(22) PCT Filed: Jun. 28, 2017
(86) PCT No.: PCT/US2017/039820

§ 371 Date: Dec. 20, 2018

Related U.S. Application Data
(60) Provisional application No. 62/507,517, filed on May 17, 2017.
(60) Provisional application No. 62/501,713, filed on May 4, 2017.
(60) Provisional application No. 62/491,150, filed on Apr. 27, 2017.
(60) Provisional application No. 62/458,455, filed on Feb. 13, 2017.
(60) Provisional application No. 62/355,846, filed on Jun. 28, 2016.

(51) Int. Cl. G01N 33/68

(52) U.S. Cl. CPC G01N 33/6878; G01N 2800/065; G01N 33/6863

(57) ABSTRACT

Provided herein are methods and compositions for evaluating a subject having or suspected of having Celiac disease.

### RELATED APPLICATIONS

This application claims the benefit of priority under 35 U.S.C. § 119(e) of U.S. provisional application No. 62/355,846, filed Jun. 28, 2016, U.S. provisional application No. 62/458,455, filed Feb. 13, 2017, U.S. provisional application No. 62/491,150, filed Apr. 27, 2017, U.S. provisional application No. 62/501,713, filed May 4, 2017, and U.S. provisional application No. 62/507,517, filed May 17, 2017, the entire contents of each of which are incorporated by reference herein.

### BACKGROUND OF THE INVENTION

Celiac disease is an autoimmune-like disorder of the small intestine that occurs in people of all ages and affects approximately 1% of people in Europe and North America. Celiac disease generally causes damage to the villi of the small intestine due to an inappropriate immune response to gluten peptides, leading to malabsorption and an increased risk of intestinal cancer. Correctly diagnosing celiac disease is important in order to ensure that those affected by celiac disease receive proper treatment.

### SUMMARY OF THE INVENTION

The disclosure relates, at least in part, to the measurement of circulating cytokines and chemokines for use in identifying subjects having or suspected of having Celiac disease or for use in determining efficacy of gluten peptide therapy.

Aspects of the disclosure relate to a method, comprising measuring a level of at least one circulating cytokine or chemokine in a subject that has or is suspected of having celiac disease, wherein the subject has been administered a single dose of a composition comprising gluten protein. In some embodiments of any one of the methods provided, the method comprises assessing the likelihood the subject has celiac disease. In some embodiments of any one of the methods provided, the method comprises assessing the efficacy of a treatment with a gluten peptide therapy. In some embodiments of any one of the methods provided, the composition comprising gluten protein contains 3 grams of gluten. In some embodiments of any one of the methods provided, the composition comprising gluten contains 6 grams of gluten. In some embodiments of any one of the methods provided, the composition comprising gluten protein is a foodstuff. In some embodiments of any one of the methods provided, the composition comprising gluten protein is a liquid composition. In some embodiments of any one of the methods provided, the composition comprising gluten protein is administered by oral administration. In some embodiments of any one of the methods provided, the composition comprising gluten protein is administered over a time period of 10 minutes or less, e.g., over a time period of 10 minutes, 9 minutes, 8 minutes, 7 minutes, 6 minutes, 5 minutes, 4 minutes, 3 minutes, 3 minutes, 2 minutes, 1 minute, or less than 1 minute. In some embodiments of any one of the methods provided, the subject is following a gluten free diet.

In some embodiments of any one of the methods provided, the method further comprises obtaining a sample from the subject and the measuring is performed on the sample. In some embodiments of any one of the methods provided, the sample from the subject is obtained 1 hour to 6 hours after the subject has been administered the single dose of the gluten protein. In some embodiments of any one of the methods provided, the sample from the subject is obtained 1 hour after the subject has been administered the single dose of the gluten protein. In some embodiments of any one of the methods provided, the sample from the subject is obtained 2 hours after the subject has been administered the single dose of the gluten protein. In some embodiments of any one of the methods provided, the sample from the subject is obtained 3 hours after the subject has been administered the single dose of the gluten protein. In some embodiments of any one of the methods provided, the sample from the subject is obtained 4 hours after the subject has been administered the single dose of the gluten protein. In some embodiments of any one of the methods provided, the sample from the subject is obtained 5 hours after the subject has been administered the single dose of the gluten protein. In some embodiments of any one of the methods provided, the sample from the subject is obtained 6 hours after the subject has been administered the single dose of the gluten protein.

In some embodiments of any one of the methods provided, the sample from the subject is a plasma or serum sample. In some embodiments of any one of the methods provided, the sample from the subject is a serum sample.

In some embodiments of any one of the methods provided, the at least one circulating cytokine or chemokine is MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 or sCD40L. In some embodiments of any one of the methods provided, the at least one circulating cytokine or chemokine is IL-8. In some embodiments of any one of the methods provided, the at least one circulating cytokine or chemokine is IL-2.

In some embodiments of any one of the methods provided, an elevated level of the at least one circulating cytokine or chemokine compared to a control level of the at least one circulating cytokine or chemokine indicates that the subject has celiac disease, and the step of assessing comprises comparing the level of the at least one circulating cytokine or chemokine to a control level of the at least one circulating cytokine or chemokine.

In some embodiments of any one of the methods provided, the control level is a baseline level. In some embodiments, the baseline level is a level of the at least one circulating cytokine or chemokine prior to administration of the composition.

In some embodiments of any one of the methods provided, the method further comprises recording whether or not the subject has celiac disease based on the assessing. In some embodiments of any one of the methods provided, the method further comprises treating, suggesting a treatment, or giving information in regard to a treatment to the subject. In some embodiments of any one of the methods provided, the treating or treatment comprises administration of a composition comprising a gluten peptide to the subject.

In some embodiments of any one of the methods provided, the subject has received treatment with a gluten peptide therapy. In some embodiments of any one of the methods provided, an elevated level of at least one circulating cytokine or chemokine compared to a control level of the at least one circulating cytokine or chemokine indicates that the subject has an unsatisfactory therapeutic response to the gluten peptide therapy, and the step of assessing comprises comparing the level of the at least one circulating cytokine or chemokine to a control level of the at least one circulating cytokine or chemokine.

In some embodiments of any one of the methods provided, measuring the level of the at least one circulating cytokine or chemokine comprises an immuno-based assay. In some embodiments of any one of the methods provided, the immuno-based assay comprises an ELISA or a multiplex bead-based assay. In some embodiments of any one of the methods provided, the immuno-based assay comprises an electrochemiluminescence assay. In some embodiments of any one of the methods provided, the assay comprises an MSD® MULTI-SPOT assay. In some embodiments of any one of the methods provided, the MSD® MULTI-SPOT assay comprises a Chemokine Panel 1 (human) kit. In some embodiments of any one of the methods provided, the MSD® MULTI-SPOT assay comprises a Proinflammatory Panel 1 (human) kit.

In some embodiments of any one of the methods provided, the method further comprises assessing whether the subject has a homozygous HLA-DQ2.5 genotype or a non-homozygous HLA-DQ2.5 genotype. In some embodiments of any one of the methods provided, the non-homozygous HLA-DQ2.5 genotype is a heterozygous HLA-DQ2.5 genotype.

In some embodiments, the subject of any one of the methods has a homozygous HLA-DQ2.5 genotype. In some embodiments, the subject of any one of the methods has a non-homozygous HLA-DQ2.5 genotype. In some embodiments, the non-homozygous HLA-DQ2.5 genotype is a heterozygous HLA-DQ2.5 genotype. In some embodiments, the subject of any one of the methods is homozygous for HLA-DQA1*05, HLA-DQB1*02, or both HLA-DQA1*05 and HLA-DQB1*02. In some embodiments, the subject of any one of the methods is not homozygous for HLA-DQA1*05, HLA-DQB1*02, or both HLA-DQA1*05 and HLA-DQB1*02.

In some embodiments of any one of the methods provided, the subject has received treatment with a gluten peptide therapy.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a table depicting an exemplary Schedule of Assessments (SOA).

FIG. 2 is a schematic depicting an exemplary 3 gram gluten bolus test for a patient on a gluten-free diet seeking diagnosis for celiac disease.

FIG. 3 is a graph showing plasma IL-8 levels measured after 3 gram gluten bolus administered to a celiac disease patients on a gluten free diet.

FIG. 4 is a graph showing plasma IL-2 levels measured after 3 gram gluten bolus administered to a celiac disease patients on a gluten free diet.

FIG. 5 is a graph showing serum IL-8 levels measured after 3 gram gluten bolus administered to a celiac disease patients on a gluten free diet.

FIG. 6 is a graph showing serum IL-2 levels measured after 3 gram gluten bolus administered to a celiac disease patients on a gluten free diet.

FIG. 7 is a schematic depicting an exemplary 6 gram gluten bolus test for a patient on a gluten-free diet seeking diagnosis for celiac disease.

FIG. 8 is a graph showing standard curves for measurement of cytokines (IL-2, IL-8, MCP-1, and IP-10) in a magnetic bead 38plex. Fold change=concentration post-dose/pre-dose.

FIG. 9 is a graph showing standard curves for measurement of cytokines (IL-2, IL-8, MCP-1, and IP-10) in ECL and magnetic bead assays. Fold change=plasma concentration post-dose/pre-dose.

FIG. 10 is a graph showing median fold change in serum cytokines after gluten 3 gram placebo-controlled challenge in CeD subjects. Blood collection was by cannula at t₀₋₈ₕ. Eotaxin 3, MCP-4, MIP-1α, TARC, IL-13, IL-12p70 are not shown as median change was <2 fold.

FIG. 11 is a graph showing median fold change in serum cytokines after gluten 3 gram placebo-controlled challenge in CeD subjects. Blood collection was by separate venipunctures. Eotaxin 3, MCP-4, MIP-1α, TARC, IL-13, IL-12p70 are not shown as median change was <2 fold.

FIG. 12 is a graph showing median-fold change in serum cytokines (IL-2 and IL-8) with gluten 6 g challenge in CeD and non-CeD subjects on a GFD. Performance CeD vs Non-CeD with optimized cutoffs: IL-2 89% sensitive, 100% specific; IL-8 74% sensitive, 100% specific.

FIG. 13 is a graph showing median-fold change in plasma cytokines with oral gluten and intradermal gluten peptide composition 150 μg i.d. first dose.

FIG. 14 is a graph showing median-fold change in a MAGPIX 38plex cytokine/chemokine assay of plasma from HLA-DQ2.5+ CeD patients on GFD administered gluten peptide composition 150 μg i.d. 0-6 h. Fold-change=concentration post-dose/pre-dose.

FIG. 15 is a schematic showing a subject with gluten sensitivity can be one that meets or can meet the algorithm.

### DETAILED DESCRIPTION OF THE INVENTION

### Diagnostic Methods

One aspect of the disclosure relates to methods of identifying (e.g., diagnosing) subjects, such as subjects having or suspected of having Celiac disease.

In some embodiments, the method comprises measuring a level of at least one circulating cytokine or chemokine in a subject that has or is suspected of having celiac disease, wherein the subject has been administered a composition comprising gluten protein as described herein. In some embodiments of any one of the methods provided, the composition comprising gluten protein contains 3 grams of gluten. In some embodiments of any one of the methods provided, the composition comprising gluten protein contains 6 grams of gluten. In some embodiments of any one of the methods provided, the composition comprising gluten protein is a foodstuff. In some embodiments of any one of the methods provided, the composition comprising gluten protein is a liquid composition. In some embodiments of any one of the methods provided, the composition comprising gluten protein is administered by oral administration. In some embodiments of any one of the methods provided, the composition comprising gluten protein is administered over a time period of 10 minutes or less, e.g., over a time period of 10 minutes, 9 minutes, 8 minutes, 7 minutes, 6 minutes, 5 minutes, 4 minutes, 3 minutes, 3 minutes, 2 minutes, 1 minute, or less than 1 minute. In some embodiments of any one of the methods provided, the composition comprising gluten protein is given as a bolus. In some embodiments of any one of the methods provided, the subject is following a gluten free diet.

In some embodiments of any one of the methods provided, the method further comprises assessing the likelihood the subject has celiac disease. In some embodiments, assessing comprises comparing the level of the at least one circulating cytokine or chemokine to a control level of the at least one circulating cytokine or chemokine. Levels as used herein can be absolute or relative amounts. In some embodiments, assessing comprises determining the ratio of the level of the at least one circulating cytokine or chemokine to the control level. In some embodiments, the control level of the at least one circulating cytokine or chemokine is a baseline level of the circulating cytokine or chemokine. In some embodiments, the baseline level is the level of the circulating cytokine or chemokine in the subject prior to the administration of the gluten protein composition. In some embodiments of any one of the methods provided herein, the method can further comprise the step of determining a baseline level of the circulating cytokine or chemokine in the subject.

In some embodiments of any one of the methods provided, an elevated level of the at least one circulating cytokine or chemokine compared to a control level, such as a baseline level, of the at least one circulating cytokine or chemokine indicates that the subject has or is likely to have celiac disease. In some embodiments, a ratio greater than 1 (e.g., greater than 2, 3, 4, 5, 6, 7, 8, 9, 10 or more) of the at least one circulating cytokine or chemokine to the control level, such as a baseline level, indicates that the subject has or is likely to have celiac disease. In some embodiments of any one of the methods provided herein, the method further comprises recording whether or not the subject has or is likely to have celiac disease based on the level or ratio.

In some embodiments of any one of the methods provided, the at least one circulating cytokine or chemokine is MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 or sCD40L. In some embodiments of any one of the methods provided, the at least one circulating cytokine or chemokine is IL-8. In some embodiments of any one of the methods provided, the at least one circulating cytokine or chemokine is IL-2.

In some embodiments of any one of the methods provided, the level of the at least one circulating cytokine or chemokine is measured in a sample, e.g., a serum, plasma or urine sample, obtained from the subject. Samples are described elsewhere herein. In some embodiments of any one of the methods provided, the sample is obtained from the subject within 1-24 hours, such as within 1-6 hours, of administration of the composition. In some embodiments of any one of the methods provided, the sample from the subject is obtained 1 hour after the subject has been administered the single dose of the gluten protein. In some embodiments of any one of the methods provided, the sample from the subject is obtained 2 hours after the subject has been administered the single dose of the gluten protein. In some embodiments of any one of the methods provided, the sample from the subject is obtained 3 hours after the subject has been administered the single dose of the gluten protein. In some embodiments of any one of the methods provided, the sample from the subject is obtained 4 hours after the subject has been administered the single dose of the gluten protein. In some embodiments of any one of the methods provided, the sample from the subject is obtained 5 hours after the subject has been administered the single dose of the gluten protein. In some embodiments of any one of the methods provided, the sample from the subject is obtained 6 hours after the subject has been administered the single dose of the gluten protein.

In some embodiments of any one of the methods provided herein, no other gluten protein compositions are provided to the subject to assess the likelihood the subject has Celiac disease. In some embodiments of any one of the methods provided herein, no other gluten protein compositions are given between the administration of the single gluten protein composition and the sample taken within 1-24 hours from the subject.

In some embodiments of any one of the methods provided, an elevated level of the at least one circulating cytokine or chemokine compared to a control level of the at least one circulating cytokine or chemokine indicates that the subject has celiac disease, and the step of assessing comprises comparing the level of the at least one circulating cytokine or chemokine to a control level of the at least one circulating cytokine or chemokine.

In some embodiments of any one of the methods provided, the control level is a baseline level. In some embodiments of any one of the methods provided, the baseline level is a level of the at least one circulating cytokine or chemokine prior to administration of the composition.

In some embodiments of any one of the methods provided, the method further comprises recording whether or not the subject has celiac disease based on the assessing. In some embodiments of any one of the methods provided, the method further comprises treating, suggesting a treatment, or giving information in regard a treatment to the subject. In some embodiments of any one of the methods provided, the treating or treatment comprises administration of a composition comprising a gluten peptide to the subject.

In some embodiments of any one of the methods provided, the subject has received treatment with a gluten peptide therapy. In some embodiments of any one of the methods provided, an elevated level of at least one circulating cytokine or chemokine compared to a control level of the at least one circulating cytokine or chemokine indicates that the subject has an unsatisfactory therapeutic response to the gluten peptide therapy, and the step of assessing comprises comparing the level of the at least one circulating cytokine or chemokine to a control level of the at least one circulating cytokine or chemokine.

In some embodiments of any one of the methods provided, measuring the level of the at least one circulating cytokine or chemokine comprises an immuno-based assay. In some embodiments of any one of the methods provided, the immuno-based assay comprises an ELISA or a multiplex bead-based assay. In some embodiments of any one of the methods provided, the immuno-based assay comprises an electrochemiluminescence assay. An exemplary electrochemiluminescence assays includes, but is not limited to, MSD-ECL Meso Scale Discovery® electrochemiluminsence (Meso Scale Diagnostics, Md.). In some embodiments of any one of the methods provided, the assay comprises an MSD® MULTI-SPOT assay. In some embodiments of any one of the methods provided, the MSD® MULTI-SPOT assay comprises a Chemokine Panel 1 (human) kit. In some embodiments of any one of the methods provided, the MSD® MULTI-SPOT assay comprises a Proinflammatory Panel 1 (human) kit.

In some embodiments of any one of the methods provided herein, the method further comprises performing other testing. Any method of other testing as described herein is contemplated. In some embodiments of any one of the methods provided, the other testing comprises a serology test, genotyping, an intestinal biopsy, and/or a T cell response test. In some embodiments of any one of the methods provided herein, the method further comprises performing one or more additional tests on the subject. In some embodiments of any one of the methods provided, the method further comprises contacting a sample comprising a T cell from the subject with a gluten peptide and measuring a T cell response in the sample. In some embodiments of any one of the methods provided, a T cell response is measured by measuring a level of IFN-γ, where an increased level of IFN-γ compared to a control level (e.g., a level of IFN-γ in a sample that has not been contacted with a gluten peptide) may identify a subject as having Celiac disease. In some embodiments of any one of the methods provided, a level of IFN-γ at or above a cut-off level (e.g., at or above 7.2 pg/ml) may identify a subject as having or likely as having Celiac disease.

In some embodiments of any one of the methods provided herein, the method further comprises treating or suggesting a treatment if the subject is identified as having or likely of having celiac disease. In some embodiments of any one of the methods provided herein, the method further comprises recommending a gluten-free diet and/or providing information in regard thereto to the subject. In some embodiments of any one of the methods provided herein, the method further comprises administering a treatment, or providing information in regard thereto, to the subject. Suitable treatments are described herein. In some embodiments of any one of the methods provided, the treatment is a composition comprising a gluten peptide as described herein. In some embodiments, the treatment comprises a gluten-free diet.

### Therapeutic Efficacy Methods

One aspect of the disclosure relates to methods of assessing the efficacy of treatment of celiac disease (e.g., responsiveness to a therapeutic gluten peptide composition). In some embodiments of any one of the methods provided, the subject has been administered a single dose of a composition comprising gluten protein, and the method comprises assessing the efficacy of a treatment with a gluten peptide therapy. In some embodiments of any one of the methods provided, the composition comprising gluten protein contains 3 grams of gluten. In some embodiments of any one of the methods provided, the composition comprising gluten protein contains 6 grams of gluten. In some embodiments of any one of the methods provided, the composition comprising gluten protein is administered by oral administration. In some embodiments of any one of the methods provided, the subject is following a gluten free diet.

In some embodiments of any one of the methods provided, assessing comprises comparing the level of the at least one circulating cytokine, chemokine, or T cell to a control level of the at least one circulating cytokine, chemokine, or T cell. Levels as used herein can be absolute or relative amounts. In some embodiments of any one of the methods provided, assessing comprises determining the ratio of the level of the at least one circulating cytokine, chemokine, or T cell to the control level. In some embodiments of any one of the methods provided, the control level of the at least one circulating cytokine, chemokine, or T cell is a baseline level of the circulating cytokine, chemokine, or T cell. In some embodiments of any one of the methods provided, the baseline level is the level of the circulating cytokine, chemokine, or T cell in the subject prior to the administration of the composition comprising gluten protein. In some embodiments of any one of the methods provided herein, the method can further comprise the step of determining a baseline level of the circulating cytokine, chemokine, or T cell in the subject.

In some embodiments of any one of the methods provided, the assessing comprises comparing the level of the at least one circulating cytokine or chemokine, and/or the level of at least one circulating T cell to a circulating cytokine or chemokine control level, such as a baseline level, and/or a circulating T cell control level, respectively. In some embodiments of any one of the methods provided, the method further comprises recording the level(s), the result(s) of the assessing and/or the treatment, or suggestion for treatment, based on the assessing.

In some embodiments of any one of the methods provided, a ratio of about 1 of the at least one circulating cytokine, chemokine, or T cell compared to a control level, such as a baseline level or negative control, of the at least one circulating cytokine, chemokine, or T cell indicates that a treatment has been effective. In some embodiments of any one of the methods provided, a ratio of greater than 1 of the at least one circulating cytokine, chemokine, or T cell compared to a control level, such as a baseline level or negative control, of the at least one circulating cytokine, chemokine, or T cell indicates that a treatment has not been effective or completely effective. In some embodiments of any one of the methods provided, a ratio of greater than or about equal to 1 of the at least one circulating cytokine, chemokine, or T cell compared to a control level, such as a positive control, of the at least one circulating cytokine, chemokine, or T cell indicates that a treatment has not been effective or completely effective. In some embodiments of any one of the methods provided, a ratio of less than 1 of the at least one circulating cytokine, chemokine, or T cell compared to a control level, such as a positive control, of the at least one circulating cytokine, chemokine, or T cell indicates that a treatment has been effective. In some embodiments of any one of the methods provided, the method further comprises recording whether or not the treatment has been effective or completely effective based on the level or ratio.

In some embodiments of any one of the methods provided, a level of the at least one circulating cytokine, chemokine, or T cell that is no more than two-fold above a control level, such as a baseline level or negative control, of the at least one circulating cytokine, chemokine, or T cell indicates that a treatment has been effective. In some embodiments of any one of the methods provided, a level of the at least one circulating cytokine, chemokine, or T cell that is two-fold or more above a control level, such as a baseline level or negative control, of the at least one circulating cytokine, chemokine, or T cell indicates that a treatment has not been effective or completely effective. In some embodiments of any one of the methods provided, a level of IL-2 and IL-8 that are each no more than two-fold above a control level, such as a baseline level or negative control, of IL-2 and IL-8 indicates that a treatment has been effective. In some embodiments of any one of the methods provided, a level of IL-2 and IL-8 that is two-fold or more above a control level, such as a baseline level or negative control, of IL-2 and IL-8 indicates that a treatment has not been effective or completely effective.

In some embodiments of any one of the methods provided, the measuring is performed on a sample obtained from the subject, e.g., a serum, plasma or urine sample. Samples are described herein. In some embodiments of any one of the methods provided, the method further comprises obtaining the sample from the subject. In some embodiments of any one of the methods provided, the sample is obtained from the subject within 4-6 hours of administration of the composition. In some embodiments of any one of the methods provided, the sample is obtained from the subject within 1-24 hours, such as within 1-6 hours, of administration of the composition. In some embodiments of any one of the methods provided, the sample from the subject is obtained 1 hour after administration of the composition. In some embodiments of any one of the methods provided, the sample from the subject is obtained 2 hours after administration of the composition. In some embodiments of any one of the methods provided, the sample from the subject is obtained 3 hours after administration of the composition. In some embodiments of any one of the methods provided, the sample from the subject is obtained 4 hours after administration of the composition. In some embodiments of any one of the methods provided, the sample from the subject is obtained 5 hours after administration of the composition. In some embodiments of any one of the methods provided, the sample from the subject is obtained 6 hours after administration of the composition.

In some embodiments of any one of the methods provided, the method further comprises administering the composition comprising gluten protein as described herein to the subject, e.g., by injection or oral administration. In some embodiments of any one of the methods provided, the composition is administered via oral administration. In some embodiments of any one of the methods provided, the composition is administered once. In some embodiments of any one of the methods provided, the composition is administered once via oral administration.

In some embodiments of any one of the methods provided, treating comprises continuing with the treatment, or suggesting comprises suggesting the subject continue with the treatment, based on the assessing. In some embodiments of any one of the methods provided, treating comprises ceasing the treatment, or suggesting comprises suggesting the subject cease the treatment, based on the assessing. In some embodiments of any one of the methods provided, treating comprises administering a different or additional treatment, or the suggesting comprises suggesting the subject be treated with an additional or different treatment, based on the assessing. Exemplary treatments are described herein. In some embodiments of any one of the methods provided, the treatment is a composition comprising a gluten peptide as described herein.

In some embodiments of any of the methods herein, the method further comprises orally administering or directing the subject to consume gluten prior to the measuring step.

In some embodiments of any one of the methods provided, the method further comprises performing other testing. Any method of other testing as described herein is contemplated. In some embodiments of any one of the methods provided, the other testing comprises a serology test, genotyping, an intestinal biopsy, and/or a T-cell response test. In some embodiments of any one of the methods provided, the method further comprises contacting a sample comprising a T cell from the subject (e.g., a whole blood sample) with a gluten peptide and measuring a T cell response in the sample. In some embodiments of any one of the methods provided, a T cell response is measured by measuring a level of IFN-γ. In some embodiments of any one of the methods provided, a decreased or similar level of IFN-γ compared to a control level (e.g., a level of IFN-γ in a sample that has not been contacted with a gluten peptide) indicates that a treatment has been effective. In some embodiments of any one of the methods provided, a level of IFN-γ below a cut-off level (e.g., below 7.2 pg/ml) indicates that a treatment has been effective. In some embodiments of any one of the methods provided, a T cell response is measured by measuring a level of IFN-γ, where an elevated level of IFN-γ compared to a control level (e.g., a level of IFN-γ in a sample that has not been contacted with a gluten peptide) indicates that a treatment has not been effective. In some embodiments of any one of the methods provided, a level of IFN-γ at or above a cut-off level (e.g., at or above 7.2 pg/ml) indicates that a treatment has not been effective.

In some embodiments of any one of the methods provided, a T cell response is measured by measuring a level of IP-10. In some embodiments of any one of the methods provided, a decreased or similar level of IP-10 compared to a control level (e.g., a level of IP-10 in a sample that has not been contacted with a gluten peptide) indicates that a treatment has been effective.

In some embodiments of any one of the methods provided, a T cell response is measured by measuring a level of IL-2. In some embodiments of any one of the methods provided, a decreased or similar level of IL-2 compared to a control level (e.g., a level of IL-2 in a sample that has not been contacted with a gluten peptide) indicates that a treatment has been effective.

Another aspect of the disclosure relates to methods of assessing tolerance to a gluten protein in a subject having Celiac disease. In some embodiments of any one of the methods provided, tolerance is a state of lessened responsiveness or non-responsiveness of the immune system to a gluten protein.

In some embodiments of any one of the methods provided, the method can be any of the methods provided herein. In one embodiment, the method comprises (a) measuring in a subject that has been administered a first composition comprising gluten protein a level of at least one circulating cytokine or chemokine; and (b) assessing the tolerance of the subject to the gluten protein based on the measuring. In some embodiments of any one of the methods provided, the subject is a subject that has previously received or is receiving treatment for Celiac disease. In some embodiments of any one of the methods provided, the treatment is a composition comprising a gluten peptide as described herein.

In some embodiments of any one of the methods provided, assessing comprises comparing the level of the at least one circulating cytokine or chemokine to a control level of the at least one circulating cytokine or chemokine. Levels as used herein can be absolute or relative amounts. In some embodiments of any one of the methods provided, assessing comprises determining the ratio of the level of the at least one circulating cytokine or chemokine to the control level. In some embodiments of any one of the methods provided, the control level of the at least one circulating cytokine or chemokine is a baseline level of the circulating cytokine or chemokine. In some embodiments of any one of the methods provided, the baseline level is the level of the circulating cytokine or chemokine in the subject prior to the administration of the gluten protein. In some embodiments of any one of the methods provided herein, the method can further comprise the step of determining a baseline level of the circulating cytokine or chemokine in the subject.

In some embodiments of any one of the methods provided, the assessing comprises comparing the level of the at least one circulating cytokine or chemokine to a circulating cytokine or chemokine control level, such as a baseline level. In some embodiments of any one of the methods provided, the method further comprises recording the level(s) or the result(s) of the assessing.

In some embodiments of any one of the methods provided, a ratio of about 2 or less (e.g., less than 2, less than 1, or less than 0.5) of the at least one circulating cytokine or chemokine compared to a control level, such as a baseline level or negative control, of the at least one circulating cytokine or chemokine indicates that the subject has been tolerized to the gluten protein. In some embodiments of any one of the methods provided, a ratio of greater than about 2 (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30) of the at least one circulating cytokine or chemokine to a control level, such as a baseline level or negative control, of the at least one circulating cytokine or chemokine indicates that the subject has not been tolerized to the gluten protein. In some embodiments of any one of the methods provided, the method further comprises recording whether or not the subject has been tolerized to a gluten protein based on the level or ratio.

In some embodiments of any one of the methods provided, the measuring is performed on a sample obtained from the subject, e.g., a serum, plasma, or urine sample. Samples are described herein. In some embodiments of any one of the methods provided, the method further comprises obtaining the sample from the subject. In some embodiments of any one of the methods provided, the sample is obtained from the subject within 1-24 hours, such as within 1-6 hours, of administration of the composition.

In some embodiments of any one of the methods provided, the method further comprises administering the composition comprising gluten protein as described herein to the subject, e.g., by injection or oral administration. In some embodiments of any one of the methods provided, the composition is administered via oral administration. In some embodiments of any one of the methods provided, the composition is administered once. In some embodiments of any one of the methods provided, the composition is administered once via oral administration.

In some embodiments of any one of the methods provided, the method further comprises treating the subject or recommending a treatment to the subject based on the assessing. In some embodiments of any one of the methods provided, treating comprises continuing with the treatment, or suggesting comprises suggesting the subject continue with the treatment, based on the assessing. In some embodiments of any one of the methods provided, treating comprises ceasing the treatment, or suggesting comprises suggesting the subject cease the treatment, based on the assessing. In some embodiments of any one of the methods provided, treating comprises administering a different or additional treatment, or the suggesting comprises suggesting the subject be treated with an additional or different treatment, based on the assessing. Exemplary treatments are described herein. In some embodiments of any one of the methods provided, the treatment is a composition comprising a gluten peptide as described herein.

In some embodiments of any of the methods herein, the method further comprises orally administering or directing the subject to consume gluten prior to the measuring step.

In some embodiments of any one of the methods provided, the method further comprises performing other testing. Any method of other testing as described herein is contemplated. In some embodiments of any one of the methods provided, the other testing comprises a serology test, genotyping, an intestinal biopsy, and/or a T cell response test. In some embodiments of any one of the methods provided, the method further comprises contacting a sample comprising a T cell from the subject (e.g., a whole blood sample) with a gluten peptide and measuring a T cell response in the sample. In some embodiments of any one of the methods provided, a T cell response is measured by measuring a level of IFN-γ. In some embodiments of any one of the methods provided, a decreased or similar level of IFN-γ compared to a control level (e.g., a level of IFN-γ in a sample that has not been contacted with a gluten peptide) may indicate that a subject has been tolerized to the gluten peptide. In some embodiments of any one of the methods provided, a level of IFN-γ below a cut-off level (e.g., below 7.2 pg/ml) may indicate that a subject has been tolerized to the gluten peptide. In some embodiments of any one of the methods provided, a T cell response is measured by measuring a level of IFN-γ, where an elevated level of IFN-γ compared to a control level (e.g., a level of IFN-γ in a sample that has not been contacted with a gluten peptide) may indicate that a subject has not been tolerized to the gluten peptide. In some embodiments of any one of the methods provided, a level of IFN-γ at or above a cut-off level (e.g., above 7.2 pg/ml) may indicate that a subject has not been tolerized to the gluten peptide.

In some embodiments of any one of the methods provided, a T cell response is measured by measuring a level of IP-10. In some embodiments of any one of the methods provided, a decreased or similar level of IP-10 compared to a control level (e.g., a level of IP-10 in a sample that has not been contacted with a gluten peptide) indicates that a subject has been tolerized to the gluten peptide.

In some embodiments of any one of the methods provided, a T cell response is measured by measuring a level of IL-2. In some embodiments of any one of the methods provided, a decreased or similar level of IL-2 compared to a control level (e.g., a level of IL-2 in a sample that has not been contacted with a gluten peptide) indicates that a subject has been tolerized to the gluten peptide.

### Samples

Samples, as used herein, refer to biological samples taken or derived from a subject, e.g., a subject having or suspected of having Celiac disease. Examples of samples include fluid samples. In some embodiments of any one of the methods provided, the sample comprises plasma, serum or urine. In some embodiments of any one of the methods provided herein, the methods comprise obtaining or providing the sample. In some embodiments of any one of the methods provided herein, the sample is obtained from the subject after administration to the subject of a composition comprising a gluten protein as described herein. In some embodiments of any one of the methods provided, the sample is obtained, e.g., at least 1, 2, 3, 4, 5, or 6 hours after administration of the composition to the subject. In some embodiments of any one of the methods provided, the sample is obtained, e.g., within 4 hours of administration of the composition. In some embodiments of any one of the methods provided, the sample is obtained, e.g., 4 hours after administration of the composition. In some embodiments of any one of the methods provided, the sample is obtained, e.g., within 24 hours or within 6 hours of administration of the composition. In some embodiments of any one of the methods provided, the sample is obtained, e.g., within 1 hour to 24 hours, within 1 hour to 6 hours, within 2 hours to 6 hours, within 2 hours to 4 hours, within 3 hours to 5 hours, within 3 hours to 6 hours, within 4 hours to 6 hours, or within 5 hours to 6 hours of administration of the composition. In some embodiments of any one of the methods provided, the sample is obtained within 4 hours to 6 hours of administration of the composition.

In some embodiments of any one of the methods provided herein, a second sample is obtained, e.g., a control sample. Controls and control samples are described herein. In some embodiments of any one of the methods provided, the second sample is obtained prior to administration of a composition comprising a gluten protein as described herein. In some embodiments of any one of the methods provided, the second sample is obtained at least, e.g., 1, 2, 3, 4, 5, 6, 12, 24, or more hours before administration of the composition. In some embodiments of any one of the methods provided, the second sample is obtained no more than 1, 2, 3, 4, 5, 6, 7, 8, 9, 10, 15, 20, 25, 30 or 60 minutes before administration of the composition. In some embodiments of any one of the methods provided, the second sample is obtained at least, e.g., 1, 2, 3, 4, 5, 6, 12, 24, or more hours before administration of the composition. In some embodiments of any one of the methods provided, additional samples are obtained, e.g., at different time points during treatment of a subject.

In some embodiments of any one of the methods provided herein, a sample is not contacted with a gluten protein or a composition comprising a gluten protein or peptide ex vivo after the sample is obtained from the subject.

### Subjects

A subject may include any subject that has or is suspected of having Celiac disease. Preferably, the subject is a human. In some embodiments of any one of the methods provided, the subject has one or more HLA-DQA and HLA-DQB susceptibility alleles encoding HLA-DQ2.5 (DQA1*05 and DQB1*02), HLA-DQ2.2 (DQA1*02 and DQB1 *02) or HLA-DQ8 (DQA1*03 and DQB1*0302). In some embodiments of any one of the methods provided, the subject is HLA-DQ2.5 positive (i.e., has both susceptibility alleles DQA1*05 and DQB1*02). In some embodiments of any one of the methods provided, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. In some embodiments of any one of the methods provided herein, the subject is on a gluten-free diet. In some embodiments of any one of the methods provided, the subject is a subject having been administered a treatment as described herein.

In some embodiments of any one of the methods provided, the subject has been on a gluten-free diet for a defined period of time or has not been on a gluten free diet prior to performance of any one of the methods described herein. In some embodiments of any one of the methods provided, the subject has been on a gluten-free diet for at least 14 days, at least 30 days, at least 60 days, or at least 90 days prior to performance of any one of the methods described herein. In some embodiments of any one of the methods provided, the subject has been on a gluten-free for at least 14 days, at least 30 days, at least 60 days, or at least 90 days prior, but no more than 1 year. In some embodiments of any one of the methods provided, the subject has been on a gluten-free diet for no more than 14 days, no more than 30 days, no more than 60 days, or no more than 90 days. In some embodiments of any one of the methods provided, the subject has been on a gluten-free diet for 14 days, 30 days, 60 days, or 90 days. In some embodiments of any one of the methods provided, the subject has not been on a gluten-free diet prior to performance of any one of the methods described herein. In some embodiments of any one of the methods provided, a subject may be determined to have been on a gluten-free diet or not on a gluten-free diet by asking the subject whether they have ingested gluten during the defined period of time. In some embodiments of any one of the methods provided, a subject may be determined to have been on a gluten-free diet or not on a gluten-free diet by performing other testing as described herein (e.g., biopsy or serology) or a T cell response assay as described herein.

A subject may be one with gluten sensitivity (also referred to as non-celiac gluten sensitivity). As used herein, a subject with gluten sensitivity exhibits one or more reactions to gluten but 1) does not have celiac disease or wheat allergy or 2) in which neither allergic nor autoimmune mechanisms against gluten are or can be identified. Generally, gluten sensitivity is not accompanied by the concurrence of anti-tTG autoantibodies or other autoimmune comorbidities and is distinct from celiac disease. Any one of the methods provided herein may be used to determine or confirm that a subject has gluten sensitivity and not celiac disease with or without the current methods of making such determination or confirmation.

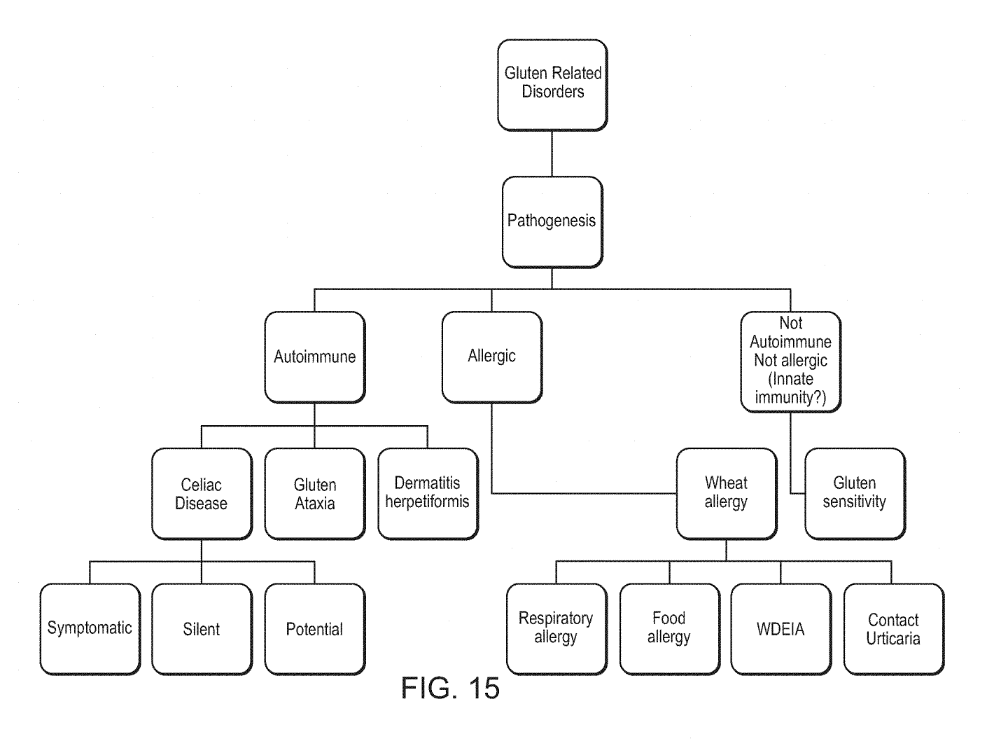

In some embodiments of any one of the methods provided herein, a subject with gluten sensitivity is one that meets or can meet the algorithm shown in FIG. 15.

In some embodiments of any one of the methods provided herein, a subject with gluten sensitivity is one that meets or can meet the Salerno Experts' Criteria (Nutrients 2015, 7, 4966-4977; doi:10.3390/nu7064966).

In some embodiments of any of the methods herein, administration comprises directly administering a composition comprising gluten to a subject. In some embodiments of any of the methods herein, administration comprises directing the subject to consume gluten prior to the measuring step.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); F. M. Ausubel et al. (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory, (1988); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Effects of Bolus Gluten Ingestion on Immune-Inflammatory Markers and Symptoms in Adults on a Gluten-Free Diet with Celiac Disease or an Intermediate Diagnosis

### Objectives:

### Primary Objective:

To evaluate the immuno-inflammatory effects of ingesting a gluten bolus compared with matched placebo in subjects with celiac disease (CeD).

### Secondary Objectives:

To evaluate the influence of HLA-DQA1*05 and HLA-DQB1*02 gene dose on the severity of symptoms and elevations of immuno-inflammatory biomarkers after gluten ingestion.
To evaluate gastro-intestinal and systemic symptoms associated with celiac disease and their temporal association to immuno-inflammatory biomarkers.
To evaluate symptoms and immuno-inflammatory biomarkers associated and with gluten ingestion in subjects who exclude dietary gluten but have not been diagnosed with CeD.

### Background:

Many patients with CeD who adhere to GFD experience gastrointestinal symptoms within hours of ingesting gluten. It is unclear what mediates these symptoms. CeD is generally associated with CD4+ T cells, B cells, and circulating IgG and IgA specific for gluten, which may be activated by gluten. IgA and IgG specific for the autoantigen, type-2 transglutaminase, are also closely associated with active CeD, but their role in the pathology of CeD is unclear. Innate immune activation has also been attributed to gluten. Activated immune cells release mediators of inflammation including cytokines and chemokines. Some cytokines, such as interleukin(IL)-2, are highly specific for distinct cell types, such as activated T cells, and others, such as complement activation are associated with antibody-mediated pathology. The present study seeks to identify biomarkers in blood that indicate the timing and quality of gluten-mediated immune activation, and whether this correlates with onset of symptoms.

An interim, unblinded analysis of an initial group of 8 subjects (6 in Cohort 1 and 2 in Cohort 2) was performed to assess optimal timing and manner of blood collection for cytokine analyses in serum and plasma. The interim analysis demonstrated (1) bolus gluten challenge with 3 g gluten was well tolerated, (2) serum was more effective than plasma for cytokine assessment, (3) cytokine elevations generally peaked at 4 h and were generally dropping 6 h after gluten ingestion, and (4) cytokine release from whole blood could be assessed equally well in both assay formats tested.

The protocol was therefore amended to (1) collection of serum alone for cytokine assessment, (2) Day 1 blood collections were limited to pre-challenge, 4 h and 6 h post-challenge using a freshly placed cannula on each occasion with serum tube collected first, (3) reducing the observation period to 6 h after challenge on Day 1, and (4) whole blood cytokine release assessed by in Quantiferon Gold Nil tubes alone.

### Patient Population:

Male and female subjects 18 to 70 years of age (inclusive) at the time of consent following a GFD for at least 1 year at the time of randomization, who have been medically diagnosed with CeD, or in whom CeD has been not been diagnosed.

### Key Inclusion Criteria:

1. Adults 18 to 70 years of age (inclusive) who have signed an informed consent form
2. For inclusion in Cohort 1, Cohort 2 or Cohort 3, a history of CeD diagnosed according to expert medical guidelines on the basis of small bowel histology showing villous atrophy and other supportive clinical and/or laboratory findings.
3. For inclusion in Cohort 4, subjects will have followed GFD but do not fulfill the expert diagnostic criteria for CeD (including those in Inclusion Criteria 1), or wheat allergy.
4. Gluten-free diet commenced at least 8-weeks prior to randomization.
5. Willingness to consume a slurry containing gluten on one occasion.

### Key Exclusion Criteria:

1. For inclusion in Cohort 1, Cohort 2 or Cohort 3, Celiac Dietary Adherence Test (CDAT, Appendix A) at screening indicates non-compliance to gluten-free diet (score >12).
2. Serum levels of both transglutaminase (tTG)-specific IgA and deamidated gliadin peptide-specific IgG are elevated. The elevation of one or other of the serology test for tTG IgA and DGP IgG is not an exclusion.
3. Subject has uncontrolled complications of celiac disease, or a medical condition which, in the opinion of the investigator, would impact the immune response or pose an increased risk to the subject.
4. Subject is or has been using an immuno-modulatory or immune suppressing medical treatment during the 2 months prior to Screening, for example azathioprine, methotrexate or biological.
5. Subject has taken oral or parenteral corticosteroids within the previous 6 weeks prior to enrollment. Topical or inhaled corticosteroids are acceptable.
6. Females who are pregnant, lactating, or breastfeeding.
7. A medical condition that may interfere with conduct of the study.

### Study Design:

This is a randomized, double-blind, placebo-controlled exploratory study to assess alterations in immune and inflammatory biomarkers, and symptoms during the six days after ingestion of gluten in adult subjects who follow a GFD who have been diagnosed with CeD or have an indeterminate diagnosis. The study will consist of an 8-week Screening Period, a single bolus ingestion of gluten or matched placebo on the morning of Day 1, and an observational period up to Day 6. With the exception of protocol-specified gluten consumption, subjects will not knowingly consume food that contains gluten throughout the study.

Subjects will be reviewed at the site 24 h and 6 days later to collect blood and monitor adverse events.

### Screening Period

Subject eligibility for randomization will be determined during a screening period of up to 8 weeks. Subjects enrolled in Cohort 1, Cohort 2 or Cohort 3 will have been diagnosed with CeD according to expert medical guidelines on the basis of small bowel histology showing villous atrophy and other supportive clinical and/or laboratory findings. Cohort 1 will include only subjects are not homozygotes for both HLA-DQA1*05 and HLA-DQB1*02. Cohort 2 will include only subjects who are homozygotes for both HLA-DQA1*05 and HLA-DQB1*02. Cohort 3 will include only subjects who do not possess both HLA-DQA1*05 and HLA-DQB1*02. Cohort 4 will include only subjects who have not been diagnosed with CeD. All subjects will have commenced GFD at least 8-weeks earlier. For enrolment in Cohort 1, Cohort 2 or Cohort 3, the Celiac Dietary Adherence Test (CDAT, Appendix A) will be performed at Visit 1. An individual will be excluded if his or her combined CDAT score is more than 12, because this score correlates with reduced compliance to GFD. Subjects will also be excluded from any Cohort if both tTG-IgA and DGP-IgG are elevated in blood collected at Visit 1, because these serologies are increased with untreated CeD.

### Gluten Challenge

Within each cohort, subjects will be randomized 2:1 to receive either gluten or a matched control. The source of gluten will be “Bobs Red Mill® Vital Wheat Gluten Flour”. The matched placebo will be gluten-free fine-ground white rice flour (“McKenzies Rice Flour”, Ward McKenzies Pty Ltd, Australia). The gluten “dose” will be 3 g, and calculated as 0.8×protein content of wheat gluten flour. For example 5 g of Bobs Red Mill® Vital Wheat Gluten Flour would be administered when the protein content (or minimum protein content if a range is stated) was 75%. The amount (weight in grams) of rice flour administered will match that of Bobs Red Mill® Vital Wheat Gluten Flour. Subjects will consume one 40 g slice of gluten free bread smeared with a single 8 g serve of butter in under 5 minutes and then immediately drink a suspension containing gluten or a matched control.

The required amount of gluten flour or rice flour will be added to one serve (7 g) of gluten-free “Vitafresh Low Calorie Lime” or “Vitafresh Low Calorie Sweet Navel Orange” (Hansells Food Group, New Zealand) and stored in the food challenge sachet in cool, dark cupboard prior to use. Prior to the challenge, 100 ml tap water at room temperature will be placed in a plastic mixing container, and following this, the sachet contents are added to the water. An electric hand blender will be used to “blitz” the contents for at least 10 seconds. The suspension is then transferred to a drink container with a lid and opening to drink from e.g. insulated coffee mug. If there is a delay between preparation and consumption, the contents should be gently and intermittently swirled to ensure the contents remain mixed. After drinking all the solution, subjects will drink up to 100 mL sparkling mineral water. Immediately afterwards, subjects will be asked whether they thought the contents contained gluten. The site investigator and subject will remain blinded to the contents of the challenge food.

### Observational Follow-Up Period

All subjects will be followed for 6 days after gluten challenge. Subjects will be observed at the study site for at least 6 h after consuming gluten on Day 1, and then return again at 24 h and 6-days post-challenge. Vital signs, adverse events, and patient reported outcomes will be recorded. Blood will be collected hourly on Day 1, and also on Day 2 and Day 6 for clinical pathology, multiplex biomarkers, gene expression, and functional and cellular markers of lymphocyte responsiveness to gluten peptides.

### Clinical Assessments:

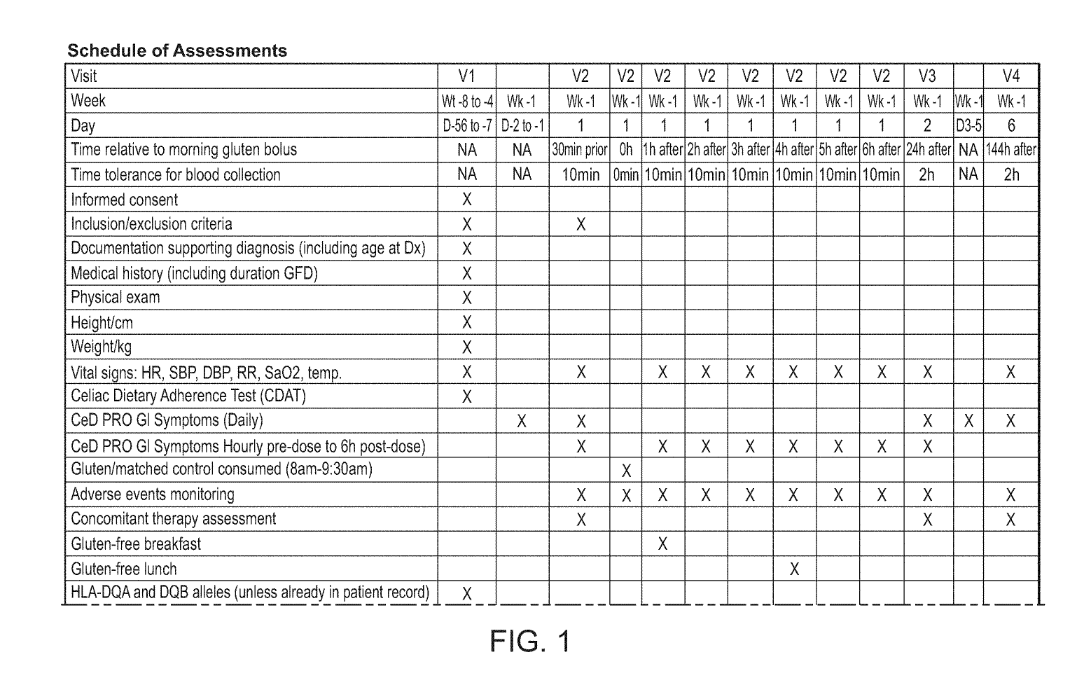

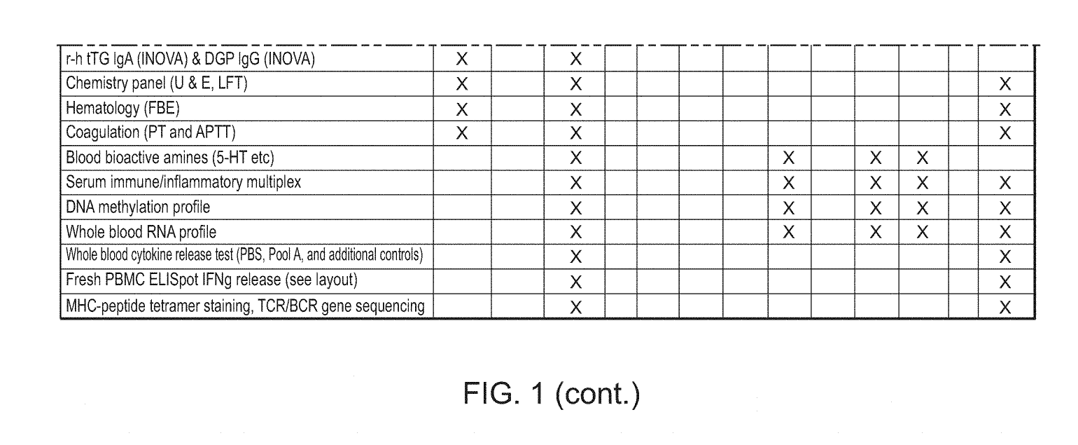

The Schedule of Assessments (SOA) is shown in FIG. 1.

Vital signs will be recorded hourly on Day 1 and include heart rate, systolic and diastolic blood pressure, respiratory rate, pulse oximetry, and body temperature.

Celiac Disease Patient Reported Outcomes tool (CeD PRO, Leffler et al 2015) relating to symptoms experienced within the previous day will be completed by subjects each morning from two days before (Day-2 and Day-1) until and including Day 6 after gluten challenge.

Celiac Disease Patient Reported Outcomes tool (CeD PRO, Leffler et al 2015) relating to symptoms experienced within the previous one hour will be completed by subjects each hour on Day 1.

At the 24 h review, the investigator will record whether they thought the subject consumed gluten or matched-placebo on Day 1.

Adverse events will be monitored continuously, and recorded at the end of each visit (Day 1, Day 2 and Day 6). The timing of onset and resolution (up to Day 6) of each adverse event will be recorded. The Investigator will assess the relationship between an AE and gluten challenge as definitely, probably, possibly, or not related. Adverse events will be graded according to “The Common Terminology Criteria for Adverse Events (CTCAE) version 4.0” (FDA Guidance for Industry—Toxicity Grading Scale).

### Clinical Laboratory Assessments:

Clinical laboratory tests (hematology, coagulation, and chemistry panels) will be taken at Visit 1 for screening, and repeated before gluten challenge on Day 1 and also on Day 6.

HLA-DQA and HLA-DQB genotype will be determined using a comprehensive panel of allele specific primers assessed at Screening Visit 1. If HLA-DQA and HLA-DQB genotype has already been established using an equivalent test and a copy is available, this earlier result is sufficient and the test will not be repeated.

CeD-specific serology tests measuring serum immunoglobulin(Ig)-A specific for human recombinant tTG-IgA (QUANTA Lite® R h-tTG IgA, INOVA Diagnostics), for Ig-G specific for deamidated gliadin peptide epitopes (QUANTA Lite® Gliadin IgG II, INOVA Diagnostics) will be taken at Screening Visit 1, and repeated before gluten challenge on Day 1.

Exploratory multiplex assessments of immuno-inflammatory biomarkers, lymphocyte stimulation assays including cytokine release assays, functional and flow cytometry-based assays of gluten-specific lymphocytes, gene-expression in whole blood and purified cell populations, such as T cells and B cells will be performed as described in the SOA in FIG. 1.

### Exploratory Immuno-Inflammatory Assessments:

Serial blood samples will be collected in suitable preservatives to enable multiplex assessments of immuno-inflammatory biomarkers, neuro-transmitters and biomarkers potentially mediating symptoms (e.g. serotonin), lymphocyte stimulation assays including cytokine release assays, functional and flow cytometry-based assays of gluten-specific lymphocytes, gene-expression in whole blood and purified cell populations, such as T cells and B cells. Timing of blood collections will be as described in the SOA.

### Sample Size:

Cohort 1 will comprise between 24 and 30 subjects.

Cohort 2 will comprise between 6 and 10 subjects.

Cohort 3 will comprise between 6 and 10 subjects.

Cohort 4 will comprise between 15 and 30 subjects.

### Statistical Methods:

No formal power calculations are performed because this is an exploratory study, however, after the 8 subjects enrolled in either Cohort 1 or Cohort 2, it is intended that two groups of 12 CeD subjects who consume gluten will provide a “Learning” followed by a “Test” set to establish and then confirm biomarkers elevated by gluten challenge.

Demographics will be presented by age, sex, duration of GFD, age at diagnosis of CeD, HLA-DQ genotype, and CeD-specific serology findings.

Change in daily CeD PRO total score, and each of the 11 separate symptom dimensions assessed from Day-2 will be presented to establish the day when symptoms were most severe following gluten or placebo challenge.

Change in hourly CeD PRO total score, and each of the 11 separate symptom dimensions assessed from pre-dose Day 1 to 6 h post-dose will be presented to establish the onset and peak severity of symptoms following gluten or placebo challenge.

Analyses of symptoms will compare Cohort 1, Cohort 2 with Cohort 3, and compare Cohort 1, 2 and 3 collectively with Cohort 4. Appropriate statistical tests will be applied for treatment comparisons to placebo at specified time points.

Adverse events (AEs) will be collected from the time subjects consume gluten or matched placebo. AEs, vital sign measurements, and clinical laboratory information will be tabulated and summarized by cohort and by treatment (active or placebo).

Analyses of AEs will compare Cohort 1, Cohort 2 with Cohort 3, and compare Cohort 1, 2 and 3 collectively with Cohort 4. Appropriate statistical tests will be applied for treatment comparisons to placebo at specified time points.

Analyses of immuno-inflammatory biomarkers will compare pre-dose with subsequent time-points. The initial phase will be directed to defining plasma biomarkers that peak within hours of gluten ingestion and correlate with onset of symptoms (e.g. 4 h), and others that peak later when symptoms are resolving (e.g. 6 h or 24 h), and others that may relate to alterations in the circulating leukocytes (e.g. gluten-specific CD4+ T cells on Day 6). For CeD subjects who consumed gluten after the initial 8 subjects, data will be divided between an initial “learning set” (the first 12 subjects consuming gluten) to establish hypotheses that will be tested in a subsequent “test set” (subsequent subjects consuming gluten). Temporal profiles of the most dynamic biomarkers will be established using the complete time-series of samples. Specificity and sensitivity of biomarkers for CeD and gluten challenge will be tested by comparing subjects with CeD who consumed gluten with others who consumed non-gluten placebo, or did not have a diagnosis of CeD. There will also be an exploratory assessment of biomarkers that might be elevated in Cohort 4 subjects who consumed gluten compared to those who consumed matched (non-gluten) placebo.

### Whole Blood Cytokine Release

Before challenge on Day 1, and on Day 6, blood will be collected from the cannula into a 10 mL syringe primed with 100 IU lithium heparin. One milliliter volumes of blood be added to Quantiferon-Gold tubes. A single aliquot of 0.1 mL PBS, and A4 (and in some cases PBS 0.1% DMSO, B4, C1, C2, CEF, or CEFT) will be added to NIL tubes, and in some cases 0.1 mL PBS will also be added to the MITOGEN tube. The cap of each tube will be replaced and then tubes will be gently inverted 3-times before being transferred to the 37° C. incubator for 24 h. At the end of the incubation period tubes are centrifuged at 2000 RCF for 10 mins RT. Plasma is removed and volumes of 0.15 mL transferred to labeled tubes. Aliquots are frozen at −80° C. before shipping for analysis.

### Example 2. Test for Patient on Gluten-Free Diet Seeking Diagnosis

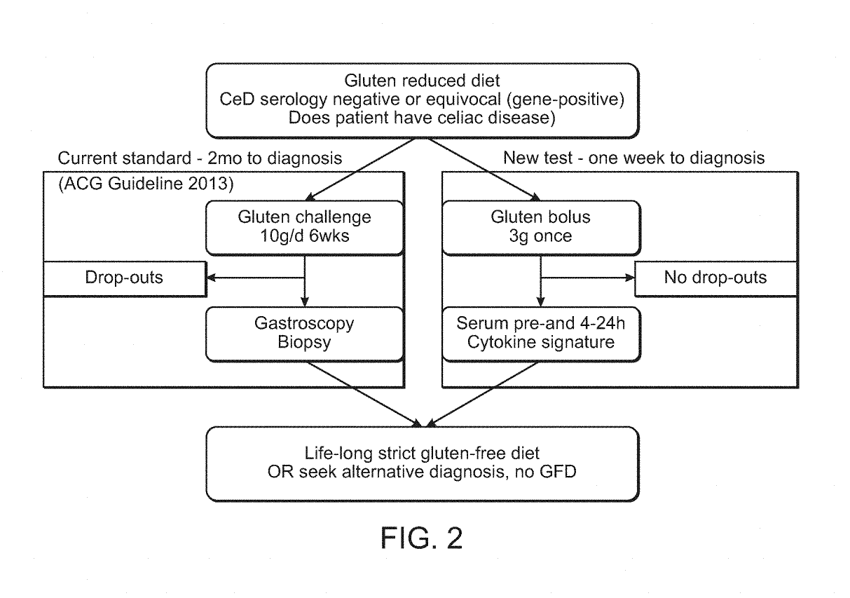

An exemplary test for a patient on a gluten-free diet seeking diagnosis is depicted in FIG. 2. A blood test following a bolus gluten ingestion as described in Example 1 was performed. No biopsy is needed and the test applied to any HLA-DQ gene.

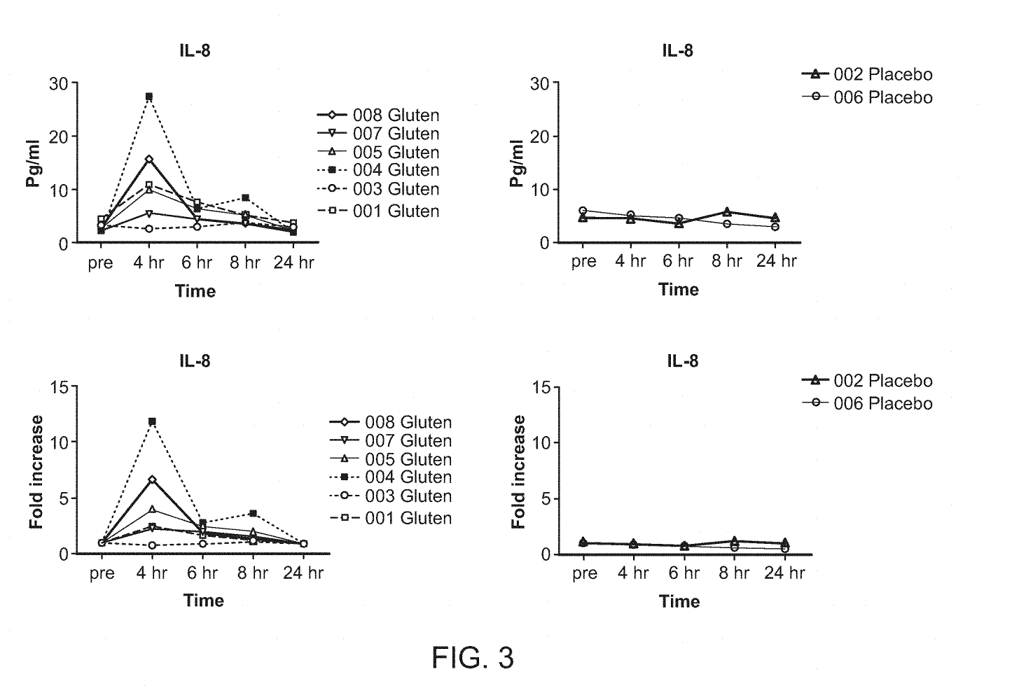

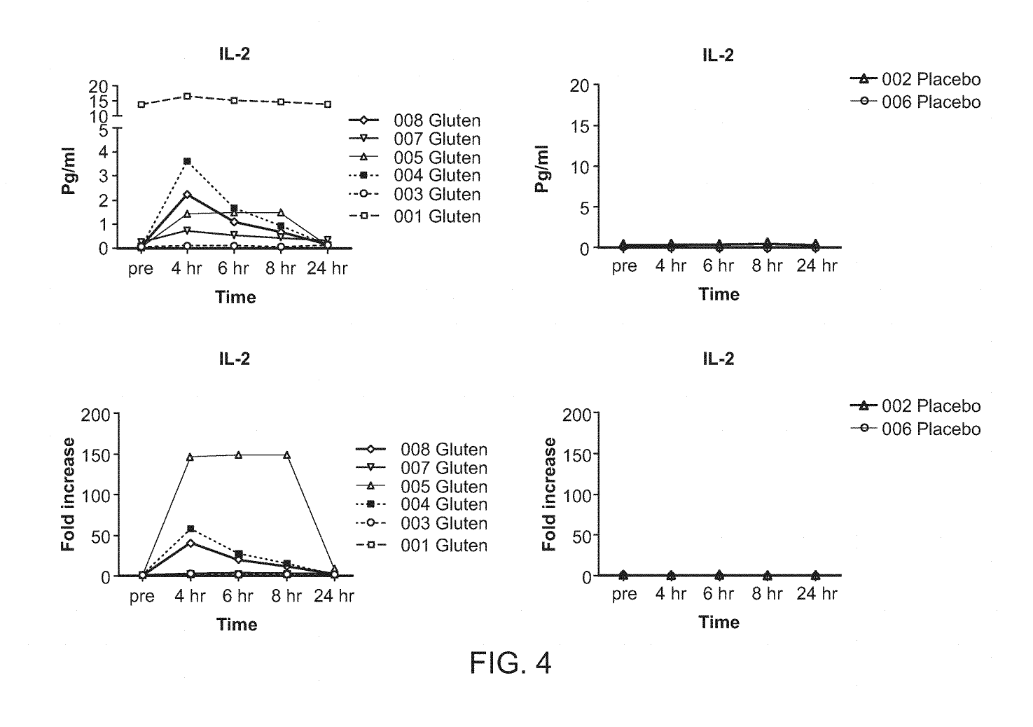

Plasma IL-8 was measured after 3 g gluten (or placebo) was administered to CeD subjects on a gluten-free diet, and the results are shown in FIG. 3. Plasma IL-2 was measured after 3 g gluten (or placebo) was administered to CeD subjects on a gluten-free diet, and the results are shown in FIG. 4.

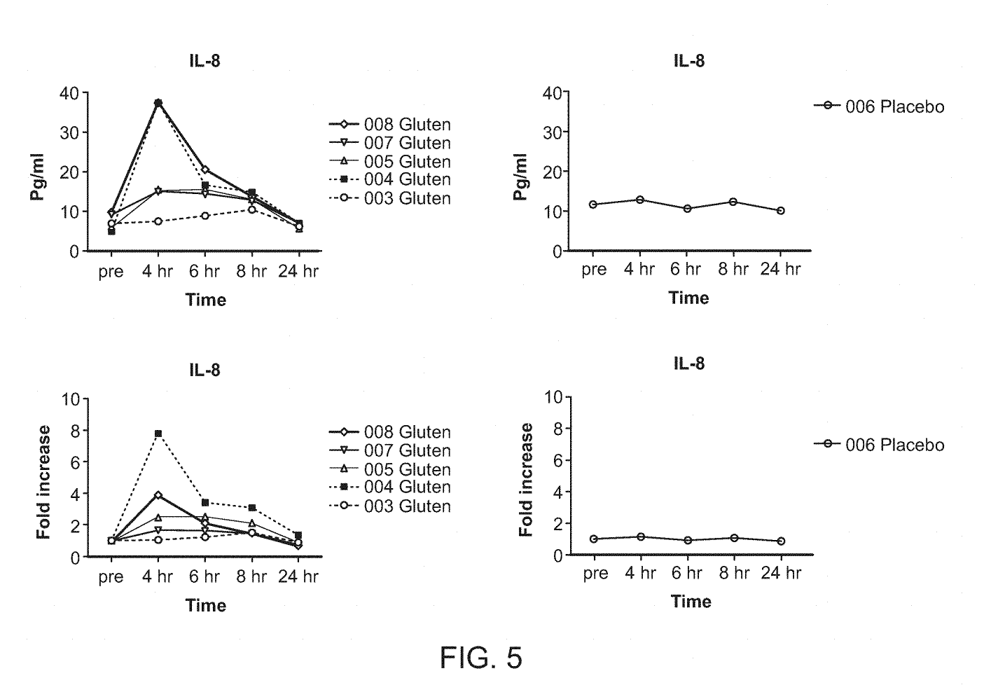

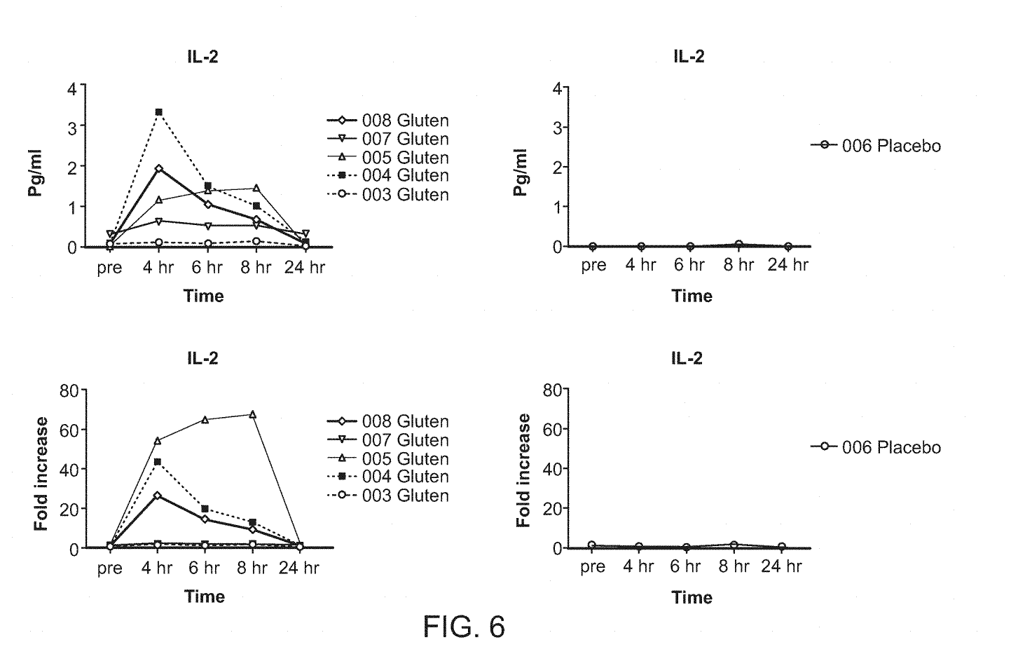

Serum IL-8 was measured after 3 g gluten (or placebo) was administered to CeD subjects on a gluten-free diet, and the results are shown in FIG. 5. Serum IL-2 was measured after 3 g gluten (or placebo) was administered to CeD subjects on a gluten-free diet, and the results are shown in FIG. 6.

### Example 3. Test for Patient on Gluten-Free Diet Seeking Diagnosis

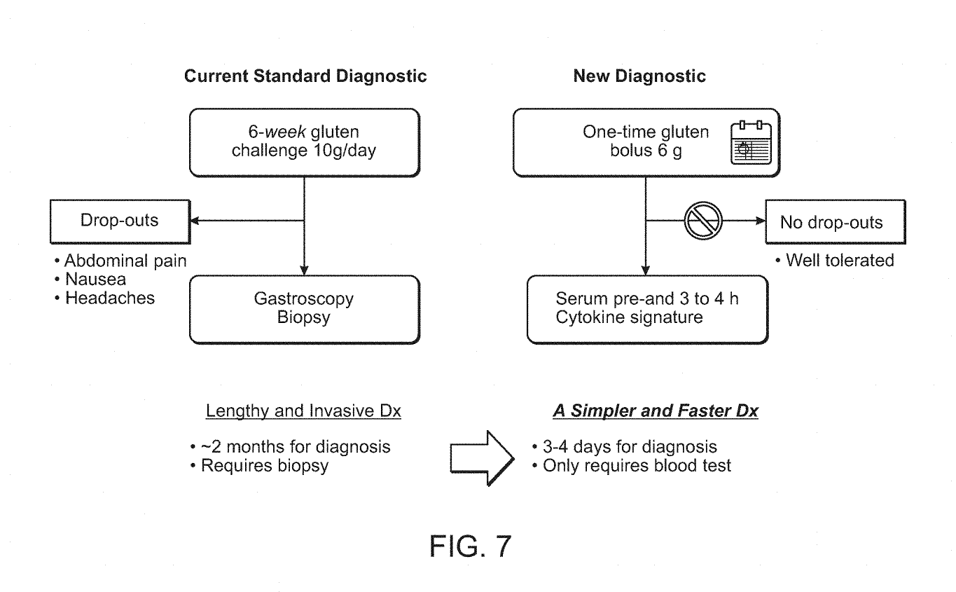

An exemplary test for a patient on a gluten-free diet seeking diagnosis is depicted in FIG. 7. A blood test following a bolus gluten ingestion of 6 grams of gluten is performed. Plasma and/or serum IL-8 is measured after 6 g gluten (or placebo) is administered to CeD subjects, or subjects suspected of having CeD, on a gluten-free diet. Plasma and/or serum IL-2 is measured after 6 g gluten (or placebo) is administered to CeD subjects, or subjects suspected of having CeD, on a gluten-free diet.

### Example 4. Gluten Ingestion and Intradermal Injection of Peptides that Activate Gluten-Specific CD4+ T Cells Elicit a Cytokine Signature Dominated by Interleukin-2 in Celiac Disease

The explanation for symptoms experienced by celiac disease (CeD) patients within hours of gluten ingestion has puzzled researchers because the early onset seems incompatible with a T-cell mediated (delayed hypersensitivity) effect. We tested the role for gluten-specific CD4+ T cells in gluten-responsive symptoms could by comparing acute immune effects of ingesting gluten with intradermal injection of Nexvax2®, an experimental adjuvant-free therapeutic vaccine that is a mixture of three short peptides including immunodominant epitopes for gluten-specific CD4+ T cells.

This example provides an exemplary assessment of changes in circulating cytokines in CeD volunteers on GFD after eating gluten or after intradermal administration of Nexvax2®.

A time course assessment of 19 cytokines and chemokines in plasma and sera from adult HLA-DQ2.5+ CeD volunteers on a GFD were compared from two separate studies. The first was a double-blind, placebo-controlled food challenge (DBPCFC) with ˜3 g wheat gluten flour compared to a matched gluten-free drink over 10 min. The second study was a double-blind, placebo-controlled phase 1 clinical trial of Nexvax2® (Goel et al. Lancet Gastro Hepatol 2017) with plasma assessed after the first dose of Nexvax2® 150 μg or normal saline i.d. in 0.1 mL. Chemokines (eotaxin, MIP-1β, eotaxin-3, TARC, IP-10, MIP-1α, MCP-1, MDC, MCP-4) and cytokines (IFN-γ, IL-1β, IL-2, IL-4, IL-6, IL-8, IL-10, IL-12p70, IL-13, TNF-α) were measured by the Meso Scale Discovery multiplex ELISA platform.

Cytokine and chemokine assessments were performed in 19 subjects after DBPCFC (11 administered gluten, & 8 non-gluten), and in 14 subjects in the final “biopsy” cohort of the phase 1 study of Nexvax2® (7 administered Nexvax2, & 7 placebo). In the first 5 subjects after gluten ingestion, hourly profiling indicated IL-2 elevation from 2 h (median 5-fold rise compared to predose, range 1.1-117) and plateauing between 3 to 5 h (median maximal fold-change 21, range 2.6-94). Median fold increase in IL-8 was between 1.8 and 2.4 from 3 to 6 h (median maximal fold-change 2.5, range 1.3-28). Median fold increases were less than 2 for other cytokines and chemokines for all time points. Overall, serum assessed from before, and at 4 and 6 h showed median IL-2 fold change of 15 at 4 h after gluten (range: 1.9-39, n=11) compared to 1.0 for non-gluten (0.5-1.6, n=8; p<0.0001 Mann Whitney). For IL-8, median fold change at 4 h after gluten was 2.4 (range: 1.0-8.4, n=11) compared to 1.1 for placebo (0.7-1.3, n=8; p=0.012 Mann Whitney). Apart from IL-2 at 6 h, no other cytokines or chemokines showed median elevation above 2-fold at any time points after gluten ingestion. After Nexvax2®, median fold changes at 2, 4 and 6 h post-dose in IL-2 were 14 (range: 1.3-68), 127 (range: 2.1-915), and 70 (range: 1.6-396), and in IL-8 were 1.6 (range: 0.9-2.8), 11 (range: 2.1-28), and 5.3 (range: 2.0-12). Weaker median fold rises of at least 2 were observed after Nexvax2® but not placebo in MCP-1, IL-10, IL-10, IFN-γ, MIP-1β, IP-10, IL-4, eotaxin, and TNFα.

IL-2, a T-cell specific cytokine, dominates the cytokine signature present between 2-6 h after gluten ingestion or intradermal administration of Nexvax2. These findings suggest ingestion of gluten or injection of peptides activate T cells rapidly, and could account for symptoms triggered by gluten.

### Example 5. Serum IL-2 and IL-8 are Elevated within 4 h after Gluten Ingestion in Celiac Disease (CeD) Patients on Gluten Free Diet (GFD)

This example provides an exemplary assessment of changes in circulating levels of cytokines and gastrointestinal symptoms in CeD volunteers on GFD after eating gluten. This example demonstrates, among other things, that gluten ingestion results in a cytokine signature in CeD patients on GFD.

### Methods:

Study 1: Randomized, Double-Blind Placebo-Controlled Challenge

˜3 gram vital gluten or gluten-free drink bolus

Subjects: HLA-DQ2.5+ CeD patients adherent to GFD

Study 2: Open Food Challenge

White bread ˜6 gram gluten over 10 minutes

Subjects: CeD patients adherent to GFD or “not CeD” on GFD supported by negative HLA-DQ genetics or histology/serology while consuming gluten

Assessments

Cytokine/chemokines in serum and/or plasma by MSD® MULTI-SPOT Assay System, Chemokine Panel 1 (human) and Proinflammatory Panel 1 (human) kits

Symptoms and safety bloods

### Results:

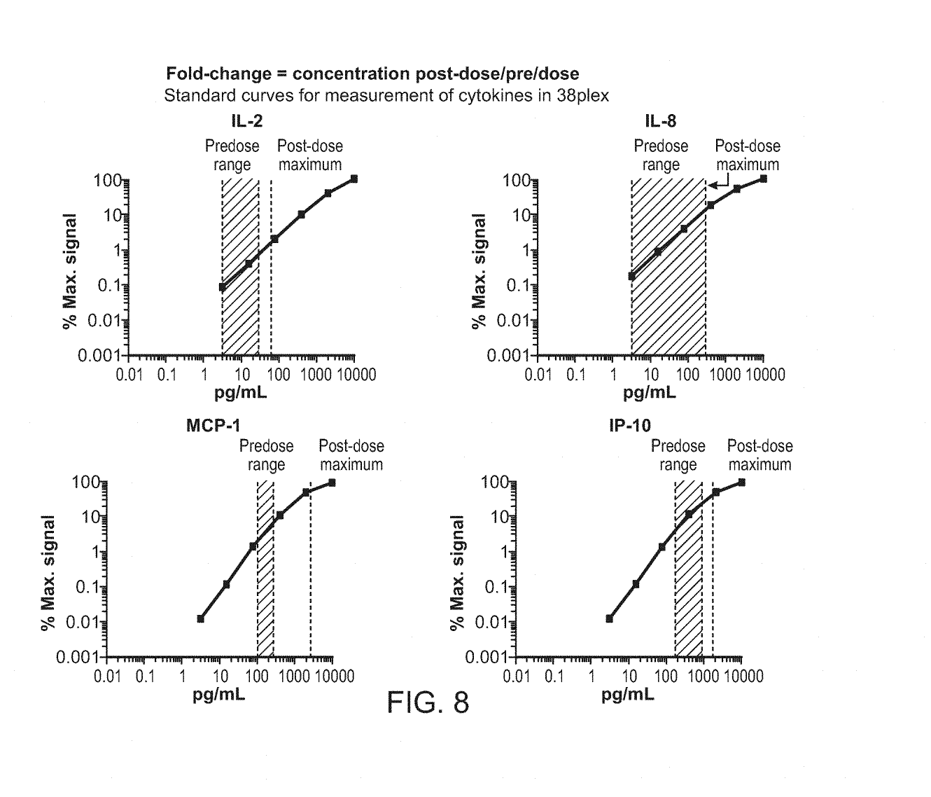

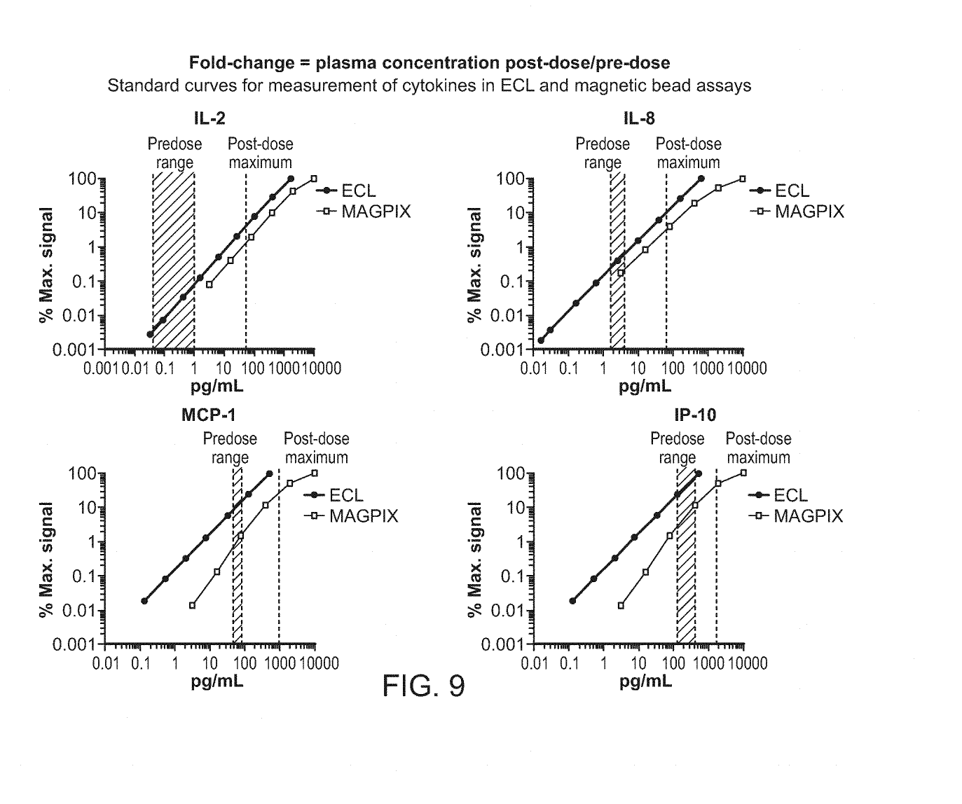

In this example, it was found that a magnetic bead 38plex may underestimate IL-2 and IL-8 fold-change in plasma after NexVax2 administration in CeD patients (FIG. 8). FIG. 8 is a graph showing standard curves for measurement of cytokines (IL-2, IL-8, MCP-1, and IP-10) in a magnetic bead 38plex. ECL 10plex assays, on the other hand, in this example, may be better suited to measurement of IL-2 and IL-8 fold-change (FIG. 9). FIG. 9 is a graph showing standard curves for measurement of cytokines (IL-2, IL-8, MCP-1, and IP-10) in ECL and magnetic bead assays.

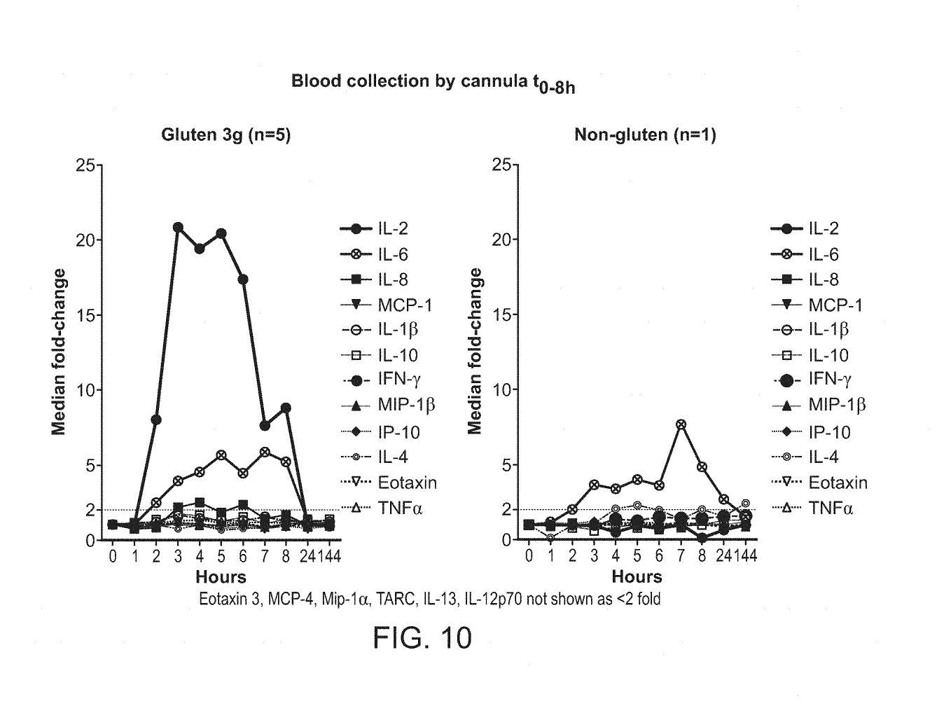

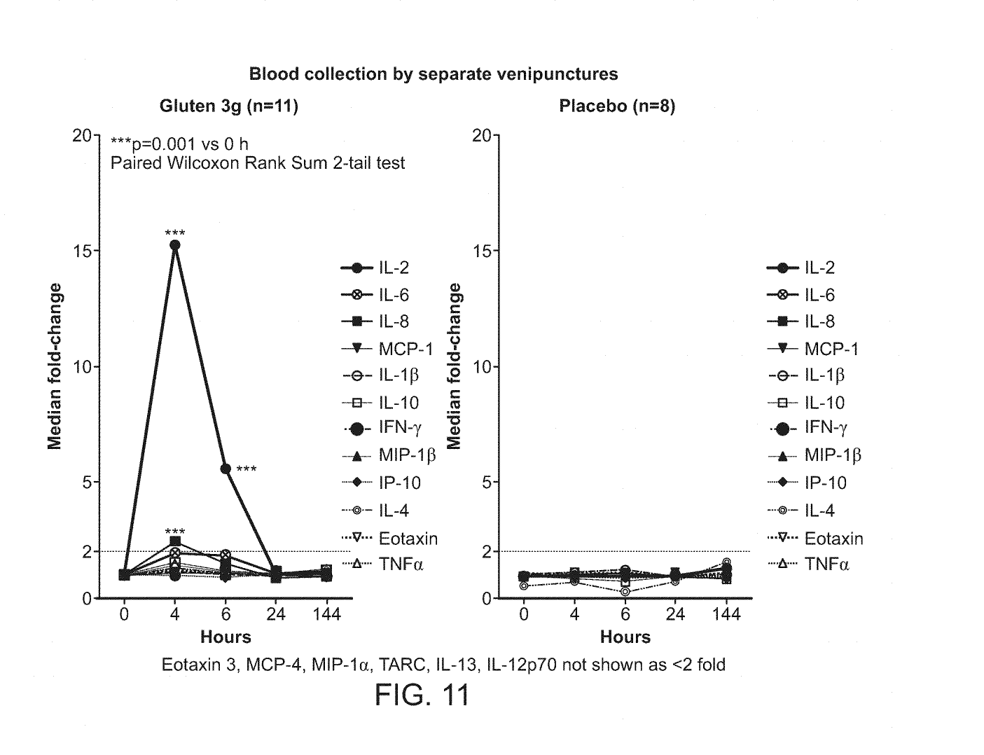

Data from Study 1 (Randomized, double-blind placebo-controlled challenge) are depicted in FIGS. 10 and 11. FIG. 10 is a graph showing median fold change in serum cytokines after gluten 3 gram placebo-controlled challenge in CeD subjects. Blood collection was by cannula at t₀₋₈ₕ. Eotaxin 3, MCP-4, MIP-1α, TARC, IL-13, IL-12p70 are not shown as median change was <2 fold. FIG. 11 is a graph showing median fold change in serum cytokines after gluten 3 gram placebo-controlled challenge in CeD subjects. Blood collection was by separate venipunctures. Eotaxin 3, MCP-4, MIP-1α, TARC, IL-13, IL-12p70 are not shown as median change was <2 fold.

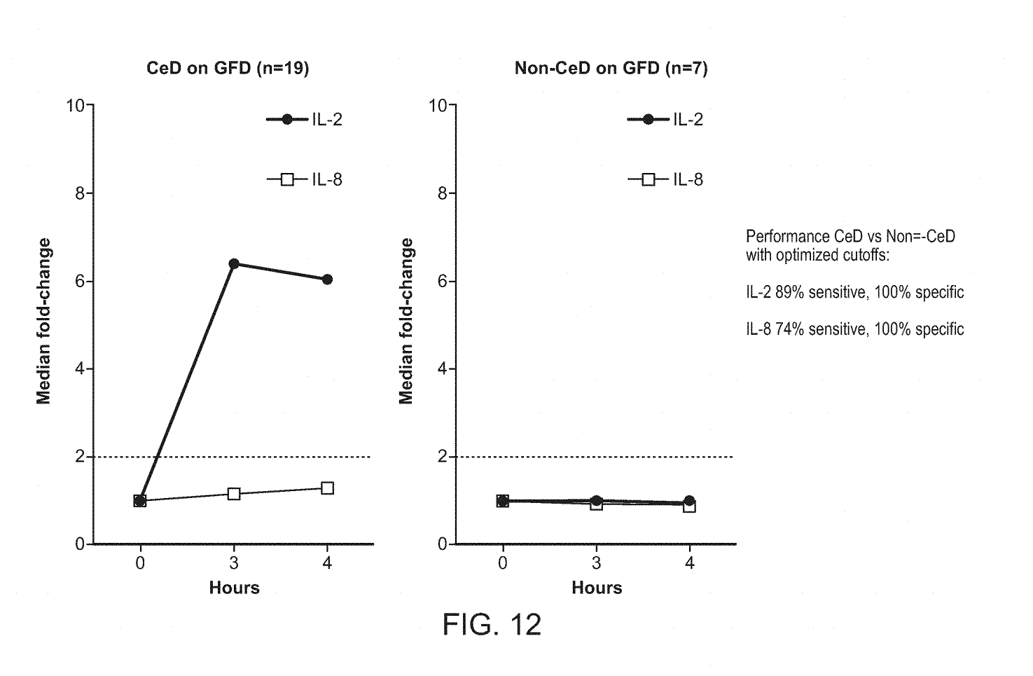

Data from Study 2 (Open food challenge) are depicted in FIG. 12. FIG. 12 is a graph showing median-fold change in serum cytokines (IL-2 and IL-8) with gluten 6 g challenge in CeD and non-CeD subjects on a GFD. Performance CeD vs Non-CeD with optimized cutoffs: IL-2 89% sensitive, 100% specific; IL-8 74% sensitive, 100% specific.

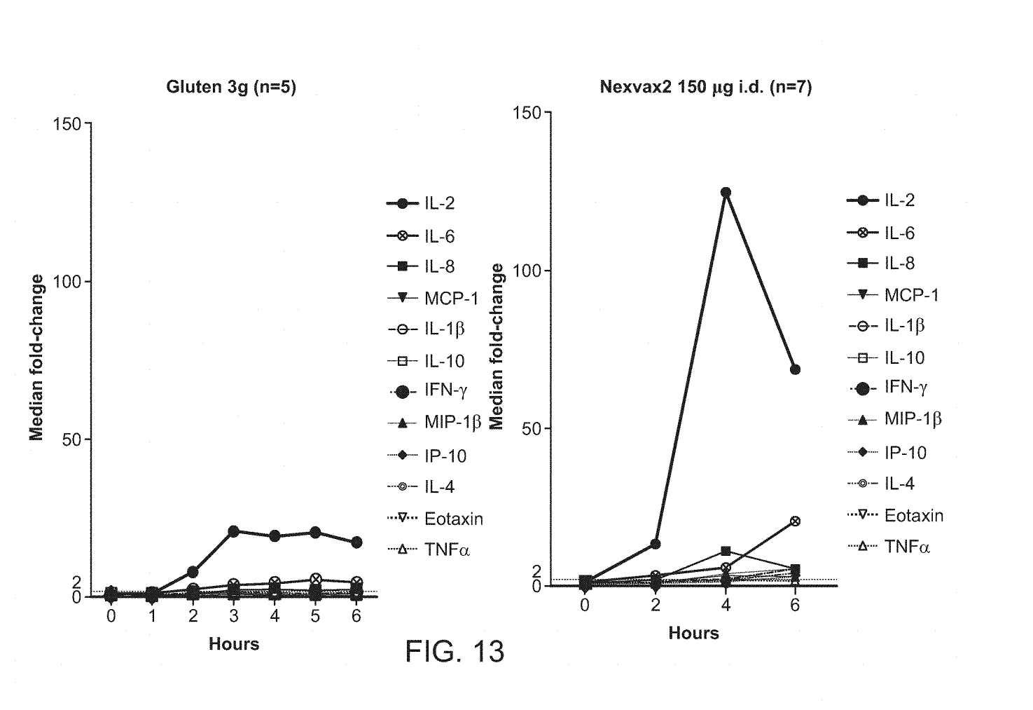

In this example, there was a quantitative but no apparent qualitative difference in CeD, as shown in FIG. 13. FIG. 13 is a graph showing median-fold change in plasma cytokines with oral gluten and intradermal NexVax2 150 μg i.d. first dose.

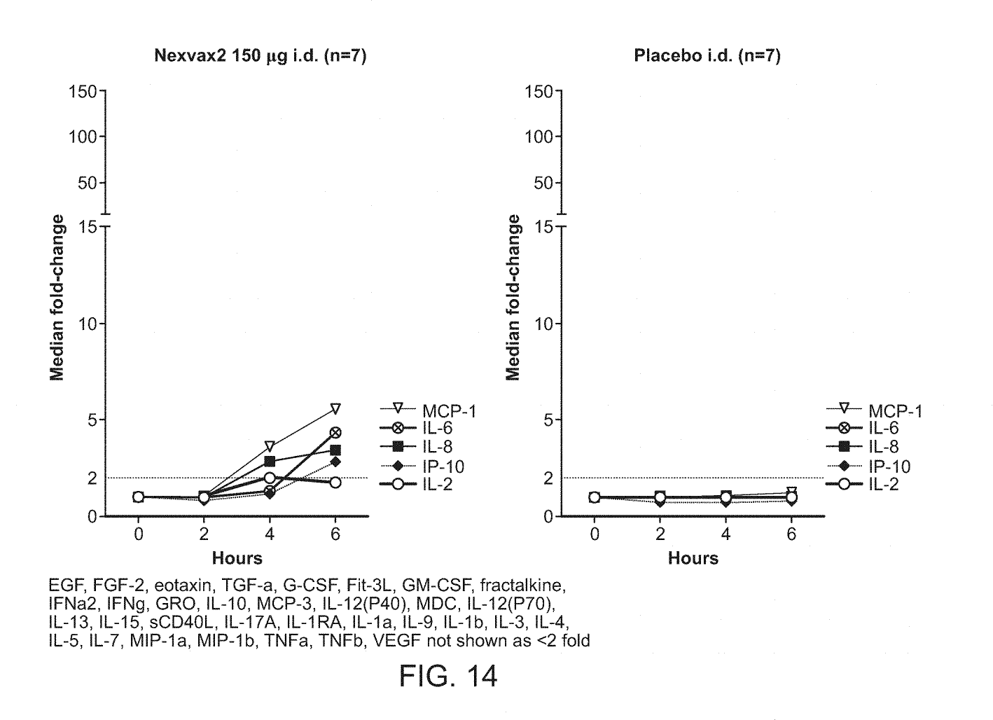

FIG. 14 is a graph showing median-fold change in a MAGPIX 38plex cytokine/chemokine assay of plasma from HLA-DQ2.5+ CeD patients on GFD administered Nexvax2 150 μg i.d. 0-6 h. Fold-change=concentration post-dose/pre-dose.

This example demonstrates, among other things, that in subjects on GFD, pronounced systemic IL-2 elevations occurred within 2 hours after gluten ingestion and appeared to be specific for CeD patients. Among 18 cytokines and chemokines associated with innate and adaptive immunity, IL-2 and IL-8 were frequently elevated. In this example, changes in blood cytokines after ingestion of gluten 3-6 g are qualitatively not different but quantitatively less than Nexvax2 150 μg i.d. in HLA-DQ2.5 CeD.

### Example 6. Elevated Plasma Interleukin(IL)-2, IL-8, and IL-10 From 2 to 6 Hours After Gluten Ingestion Differentiates Between Celiac Disease (CeD) and Non-Celiac Gluten Sensitivity (NCGS) in Patients on Gluten-Free Diet (GFD)

Gluten challenge elevates circulating cytokines in CeD patients on GFD, and IL-2, IL-8 and IL-10 were evaluated in plasma after gluten ingestion in CeD and NCGS subjects on GFD using highly sensitive assays as follows.

### Methods

Plasma was collected before, and 2, 4 and 6 hours after participants with CeD (n=19) or NCGS (n=49) on GFD consumed gluten (5.7 grams) during their participation in one of two studies. Plasma cytokines were measured by Mesoscale V-plex assays. Daily symptoms were measured by GSRS-IBS. CeD subjects were assessed for serology, duodenal histology, and frequency of gluten-specific T cells in blood using HLA-DQ:gluten tetramers.

### Results

IL-2 fold changes from pre-challenge were significantly increased in CeD compared to NCGS after gluten challenge at 2 hours (CeD median: 1.2, 25th-75th percentiles 1.0-2.6; NCGS: 1.0, 0.94-1.0; Mann Whitney U test p=0.0012), 4 hours (CeD: 10.0, 2.0-25.6; NCGS: 1.0, 0.94-1.0; p<0.0001) and 6 hours (CeD: 3.6, 1.7-18.6; NCDS: 1.0, 0.96-1.1; p<0.001). Elevations in IL-8 and IL-10 were also significantly increased in CeD compared to NCGS at 4 hours and 6 hours (p<0.0001). Median elevations were between 1.2 to 1.8-fold.

Sensitivity and specificity with optimized cutoffs for IL-2 were 74% and 98%, respectively, for IL-8 were 42% and 100%, and IL-10 were 32% and 100%. Average IL-2 fold change at 2 to 6 hours was correlated with baseline tetramer+ cells in HLA-DQ2.5+ CeD (n=16; p=0.0028, r=0.70), and overall discomfort at 6 hours (n=19; p=0.0446, r=0.49).

It was found that elevated IL-2, IL-8 and IL-10 after gluten ingestion is specific for CeD, with IL-2 being most sensitive in these studies. Measurement of circulating cytokines can assist in differentiating between CeD and NCGS.

1. A method, comprising measuring a level of at least one circulating cytokine or chemokine in a subject that has or is suspected of having celiac disease, wherein the subject has been administered a single dose of a composition comprising gluten protein, and assessing the likelihood the subject has celiac disease or assessing the efficacy of a treatment with a gluten peptide therapy.

2. The method of claim 1, wherein the composition comprising gluten protein contains 3 grams of gluten.

3. The method of claim 1, wherein the composition comprising gluten protein contains 6 grams of gluten.

4. The method of any one of claims 1-3, wherein the composition comprising gluten protein is a foodstuff.

5. The method of any of claims 1-4, wherein the composition comprising gluten protein is a liquid composition.

6. The method of any one of claims 1-5, wherein the composition comprising gluten protein is administered by oral administration.

7. The method of any one of claims 1-6, wherein the composition comprising gluten protein is administered over a time period of 10 minutes or less.

8. The method of any one of claims 1-7, wherein the subject is following a gluten free diet.

9. The method of any one of claims 1-8, wherein the method further comprises obtaining a sample from the subject and the measuring is performed on the sample.

10. The method of any one of claims 1-9, wherein the sample from the subject is obtained 1 hour to 6 hours after the subject has been administered the single dose of the gluten protein.

11. The method of any one of claims 1-10, wherein the sample from the subject is obtained 2 hours after the subject has been administered the single dose of gluten protein.

12. The method of any one of claims 1-10, wherein the sample from the subject is obtained 3 hours after the subject has been administered the single dose of the gluten protein.

13. The method of any one of claims 1-10, wherein the sample from the subject is obtained 4 hours after the subject has been administered the single dose of the gluten protein.

14. The method of any one of claims 1-10, wherein the sample from the subject is obtained 6 hours after the subject has been administered the single dose of the gluten protein.

15. The method of any one of claims 1-14, wherein the sample from the subject is a plasma or serum sample.

16. The method of any one of claims 1-15, wherein the sample from the subject is a serum sample.

17. The method of any one of claims 1-16, wherein the at least one circulating cytokine or chemokine is MCP-1, IP-10, IL-6, IL-8, G-CSF, IL-2, IL-1RA, GRO, EOTAXIN, GM-CSF, IL-10, TNFa, IFNa2, MIP-1b, IL-12P70, IL-1a, IL-17A, EGF, MIP-1a, FRACTALKINE, IFNg, VEGF, IL-9, FGF-2, IL-1b, Flt-3L, I-15, TNFb, IL-12(P40), MCP-3, IL-4, MDC, IL-13, TGF-a, IL-3, IL-5, IL-7 or sCD40L.

18. The method of any one of claims 1-16, wherein the at least one circulating cytokine or chemokine is IL-10.

19. The method of any one of claims 1-16, wherein the at least one circulating cytokine or chemokine is IL-8.

20. The method of any one of claims 1-16, wherein the at least one circulating cytokine or chemokine is IL-2.

21. The method of any one of claims 1-20, wherein an elevated level of the at least one circulating cytokine or chemokine compared to a control level of the at least one circulating cytokine or chemokine indicates that the subject has celiac disease, and the step of assessing comprises comparing the level of the at least one circulating cytokine or chemokine to a control level of the at least one circulating cytokine or chemokine.

22. The method of any one of claims 1-21, wherein the control level is a baseline level.

23. The method of claim 22, wherein the baseline level is a level of the at least one circulating cytokine or chemokine prior to administration of the composition.

24. The method of any one of claims 1-23, wherein the level as compared to a baseline level that is indicative of celiac disease is any one of the fold changes, or greater, as provided herein.

25. The method of any one of claims 1-24, wherein the method further comprises recording whether or not the subject has celiac disease based on the assessing.

26. The method of any one of claims 1-25, wherein the method further comprises treating, suggesting a treatment, or giving information in regard to a treatment to the subject.

27. The method of any one of claims 1-26, wherein the treating or treatment comprises administration of a composition comprising a gluten peptide to the subject.

28. The method of any one of claims 1-20, wherein the subject has received treatment with a gluten peptide therapy.

29. The method of claim 28, wherein an elevated level of at least one circulating cytokine or chemokine compared to a control level of the at least one circulating cytokine or chemokine indicates that the subject has an unsatisfactory therapeutic response to the gluten peptide therapy, and the step of assessing comprises comparing the level of the at least one circulating cytokine or chemokine to a control level of the at least one circulating cytokine or chemokine.

30. The method of any one of claims 1-29, wherein measuring the level of the at least one circulating cytokine or chemokine comprises an immuno-based assay.

31. The method of claim 30, wherein the immuno-based assay comprises an ELISA or a multiplex bead-based assay.

32. The method of claim 30, wherein the immuno-based assay comprises a electrochemiluminsence assay.

33. The method of any one of claims 1-29, wherein the assay comprises an MSD® MULTI-SPOT assay.

34. The method of claim 33, wherein the MSD® MULTI-SPOT assay comprises a Chemokine Panel 1 (human) kit.

35. The method of claim 33, wherein the MSD® MULTI-SPOT assay comprises a Proinflammatory Panel 1 (human) kit.

36. The method of claim 30, wherein the immune-based assay comprises a Mesoscale V-plex assay.

37. The method of any one of claims 1-36, wherein the method further comprises assessing whether the subject has a homozygous HLA-DQ2.5 genotype or a non-homozygous HLA-DQ2.5 genotype.

38. The method of claim 37, wherein the non-homozygous HLA-DQ2.5 genotype is a heterozygous HLA-DQ2.5 genotype.

39. The method of any one of claims 1-37, wherein the subject has a homozygous HLA-DQ2.5 genotype.

40. The method of any one of claims 1-37, wherein the subject has a non-homozygous HLA-DQ2.5 genotype.

41. The method of claim 37, wherein the non-homozygous HLA-DQ2.5 genotype is a heterozygous HLA-DQ2.5 genotype.

42. The method of any one of claims 1-41, wherein the subject is homozygous for HLA-DQA1*05, HLA-DQB1*02, or both HLA-DQA1*05 and HLA-DQB1*02.

43. The method of any one of claims 1-41, wherein the subject is not homozygous for HLA-DQA1*05, HLA-DQB1*02, or both HLA-DQA1*05 and HLA-DQB1*02.

44. The method of any one of claims 1-43, wherein the subject has received treatment with a gluten peptide therapy.

45. The method of any one of claims 1-43, wherein the subject has celiac disease.

46. The method of any one of claims 1-43, wherein the subject is suspected of having celiac disease.

47. The method of any one of claims 1-43, wherein the subject has gluten sensitivity.

48. The method of any one of claims 1-47, wherein the method further comprises treating, suggesting a treatment, or giving information in regard to a treatment that is non-celiac treating or treatment to the subject.

49. The method of any one of claims 1-47, wherein the method does not comprise treating, suggesting a treatment, or giving information in regard to a treatment that is celiac treating or treatment to the subject or comprises not suggesting or giving information in regard to a celiac treatment to the subject.

50. One or more compositions comprising a gluten protein for use in carrying out a method of any one of claims 1-49.

51. A kit for use in carrying out a method of any one of claims 1-49 comprising any one or more compositions provided herein.
